(19) United States
(12) Patent Application Publication
(10) Pub. No.: US 2025/0241968 A1
(43) Pub. Date: Jul. 31, 2025

(54) COMPOSITIONS AND METHODS FOR TREATING NEURODEGENERATION

(71) Applicant: The Trustees of Columbia University in the City of New York, New York, NY (US)

(72) Inventor: Umrao MONANI, Oradell, NJ (US)

(73) Assignee: The Trustees of Columbia University in the City of New York, New York, NY (US)

(21) Appl. No.: 19/064,902

(22) Filed: Feb. 27, 2025

Related U.S. Application Data
(63) Continuation of application No. PCT/US23/73459, filed on Sep. 5, 2023.
(60) Provisional application No. 63/403,475, filed on Sep. 2, 2022.

(51) Int. Cl. A61K 35/76; A61K 38/17; A61K 45/06; A61K 48/00; C07K 14/47; C12N 15/113; C12N 15/86

(52) U.S. Cl. CPC A61K 35/76; A61K 38/17; A61K 45/06; A61K 48/005; C07K 14/47; C12N 15/113; C12N 15/86; C12N 2750/14143

(57) ABSTRACT

The present disclosure provides compositions and methods of treating or preventing neurodegenerative diseases, including tau-related diseases or tauopathies. A variant or mutant of a heat shock protein, such as an Hsp70 family member protein, may be used in the present method. Alternatively, a modulator of a heat shock protein may be used.

### CROSS REFERENCE TO RELATED APPLICATION

The present application claims priority to International Patent Application No. PCT/US2023/073459 filed Sep. 5, 2023, which claims priority to U.S. Provisional Patent Application No. 63/403,475, filed Sep. 2, 2022, each of which is incorporated herein by reference in its entirety.

### STATEMENT OF GOVERNMENT SUPPORT

This invention was made with government support under grant NS104218 awarded by the National Institute of Neurological Disorders and Stroke. The government has certain rights in the invention.

### INCORPORATION-BY-REFERENCE OF SEQUENCE LISTING

A sequence listing, filed as the XML file “01001_011353-US1_SL.xml” which was created on Aug. 31, 2023 and is 3,667 bytes in size, is incorporated herein by reference in its entirety.

### FIELD OF THE INVENTION

The present invention relates to compositions and methods for treating neurodegenerative diseases.

### BACKGROUND

Homozygous loss of the survival motor neuron 1 (SMN1) gene triggers the neuromuscular disorder, spinal muscular atrophy (SMA).¹⁻³ A paralogue, SMN2, is invariably present in patients but fails to counter SMN1 loss owing to a splice-switching exon 7 transition that renders the copy gene unable to produce adequate SMN protein.^{4.5} Still, SMN2 is an intuitively appealing therapeutic target for the treatment of SMA. Indeed, nusinersen and risdiplam, two agents currently approved for SMA therapy, mitigate disease by inducing SMN2 to express greater amounts of intact SMN. A third agent, onasemnogene abeparvovec, restores SMN by means of an AAV9 vector. These therapies, if delivered in a timely manner, effectively prevent disease onset.⁶ Yet, developing these therapeutic modalities did not address our understanding of how SMN governs neuromuscular health. SMA mechanisms have instead been gleaned primarily from studies that investigated SMN functions. These have implicated SMN in diverse roles including, notably, functions in RNA metabolism.⁷ However, a compelling link between such functions and the selective vulnerability of the neuromuscular system in SMA is yet to emerge.

One approach to defining disease-relevant mechanisms involves the identification of genetic modifiers.^{8.9} Indeed, such a strategy has been pursued for SMA.^{10,11} Yet, what has emerged, especially from genetic screens carried out in invertebrates, has rarely been confirmed in mammalian models. Besides, the search for SMA modifiers in worms, flies, and even rodents is hampered by the absence of a true, SMN2-like hypomorph in these model systems. Attempts to identify SMA modifiers by studying families with discordantly affected sibs have been more fruitful.¹²⁻¹⁵ However, perplexingly, expressing the genes that emerged from these studies in humanized, SMN2-expressing mice failed to modify disease without first augmenting the mutants with an SMN-enhancing agent. Moreover, none of the putative modifiers was ever linked to a distinct genomic alteration.¹²⁻¹⁴ Consequently, discerning precisely why the identified modifiers, which are indistinguishable in modified and unmodified subjects, alter disease is difficult.

Studies indicate that the SMN protein also plays a role in the pathogenesis of other motor neuron diseases such as amyotrophic lateral sclerosis (ALS). Recent work has indicated there are genetic and etiological similarities between SMA and ALS, namely, a disruption of RNA processing appears to be involved in both. These findings open up the possibility of a common treatment strategy for SMA and ALS (Gama-Carvalho et al., Linking amyotrophic lateral sclerosis and spinal muscular atrophy through RNA-transcriptome homeostasis: a genomics perspective, J Neurochem. 2017, 141(1): 12-30).

Due to the ever-growing population of aging individuals that fall victim to tau-related dementias, there is an urgent unmet medical need to find effective ways of addressing the health concerns of these patients.

### SUMMARY

The present disclosure provides for a method of treating a neurodegenerative disease in a subject. The present disclosure provides for a method of treating a tau-related disease or tauopathy in a subject. The present disclosure provides for a method of treating a neurodegenerative dementia, or a tau-related dementia in a subject. The present disclosure provides for a method of treating Alzheimer's disease, Parkinson's disease, amyotrophic lateral sclerosis, multiple sclerosis, Huntington's disease, multiple system atrophy, Batten disease, or a prion disease in a subject. The present disclosure provides for a method of potentiating or enhancing neurotransmission.

The method may comprise administering an effective amount of a nucleic acid molecule encoding a variant, mutant or modulator of an Hsp70 family member protein (e.g., Hspa8) to the subject.

The method may comprise administering an effective amount of a variant, mutant or modulator of an Hsp70 family member protein (e.g., Hspa8) to the subject.

The variant of Hspa8 may be Hspa8^{G470R}.

The mutant Hsp70 family member protein (e.g., a mutant Hspa8) may comprise a missense mutation. The mutant Hsp70 family member protein (e.g., a mutant Hspa8) may comprise a mutation in a substrate binding domain of the Hsp70 family member protein (e.g., Hspa8). The mutant Hsp70 family member protein (e.g., a mutant Hspa8) may comprise a mutation in an ATPase domain of the Hsp70 family member protein (e.g., Hspa8).

The variant or mutant of an Hsp70 family member protein (e.g., Hspa8) may have a lower chaperone activity than the wildtype Hsp70 family member protein (e.g., wildtype Hspa8). The variant or mutant of an Hsp70 family member protein (e.g., a mutant Hspa8) may have a greater microautophagy activity than the wildtype Hsp70 family member protein (e.g., wildtype Hspa8).

The modulator may bind to a substrate binding domain of an Hsp70 family member protein (e.g., Hspa8). The modulator may bind to an ATPase domain of an Hsp70 family member protein (e.g., Hspa8).

The modulator may decrease a chaperone activity of an Hsp70 family member protein (e.g., Hspa8). The modulator may increase a microautophagy activity of an Hsp70 family member protein (e.g., Hspa8).

In one embodiment, the modulator is an inhibitor of an Hsp70 family member protein (e.g., Hspa8).

The modulator may be a small molecule, a polynucleotide (e.g., a small interfering RNA (siRNA) or an antisense molecule), or an antibody or antigen-binding portion thereof. The modulator may comprise a CRISPR/Cas system.

The variant, mutant or modulator may be administered to the central nervous system (CNS) of the subject. The variant, mutant or modulator may be administered to the spinal cord of the subject. The variant, mutant or modulator may be administered by intrathecal injection. The variant, mutant or modulator may be administered orally, intravenously, intramuscularly, topically, arterially, or subcutaneously.

The nucleic acid molecule may comprise a recombinant adeno-associated virus (AAV) vector, such as AAV-PHP.eB, AAV9 or any other AAV as described herein.

The nucleic acid molecule may be administered to the central nervous system (CNS) of the subject. The nucleic acid molecule may be administered to the spinal cord of the subject. The nucleic acid molecule may be administered by intrathecal injection. The nucleic acid molecule may be administered orally, intravenously, intramuscularly, topically, arterially, or subcutaneously.

The method may further comprise administering an SMN2 splicing modifier to the subject.

The neurodegenerative disease may be a tau-related disease or tauopathy. The tau-related disease or tauopathy may be Alzheimer's disease (AD), primary age-related tauopathy (PART) dementia, chronic traumatic encephalopathy (CTE), progressive supranuclear palsy (PSP), corticobasal degeneration (CBD), frontotemporal dementia and parkinsonism linked to chromosome 17 (FTDP-17), vacuolar tauopathy, lytico-bodig disease (Parkinson-dementia complex of Guam), Ganglioglioma and gangliocytoma, meningioangiomatosis, postencephalitic parkinsonism, subacute sclerosing panencephalitis (SSPE), lead encephalopathy, tuberous sclerosis, pantothenate kinase-associated neurodegeneration, lipofuscinosis, Pick's disease, corticobasal degeneration, argyrophilic grain disease (AGD), spinal muscular atrophy (SMA) or amyotrophic lateral sclerosis (ALS).

The neurodegenerative disease may be amyotrophic lateral sclerosis, multiple sclerosis, Parkinson's disease, Alzheimer's disease, Huntington's disease, multiple system atrophy, Batten disease, or a prion disease.

The neurodegenerative disease may be a neurodegenerative dementia.

The neurodegenerative disease may be a tau-related dementia.

The subject may be a mammal, such as a human, a rodent, or a simian.

### BRIEF DESCRIPTION OF THE DRAWINGS

For the purpose of illustrating the invention, there are depicted in drawings certain embodiments of the invention. However, the invention is not limited to the precise arrangements and instrumentalities of the embodiments depicted in the drawings.

FIGS. 1A-1I. Evidence and mapping of a C57BL/6-derived modifier of SMA in model mice. (FIG. 1A) Kaplan-Meier survival curves depicting altered lifespans of model mice on pure or hybrid strain backgrounds. p<0.0001 between C57BL/6 and FVB/N mutants, log-rank test. (FIG. 1B) Weight curves of pure and hybrid strain SMA mutants. ***, p<0.001, PND2-PND12 between FVB/N and SMA-Mod mutants, t tests. PND: postnatal day. (FIGS. 1C and 1D) Reduced disease severity in SMA-Mod mutants relative to FVB/N-derived mutants, as assessed by (FIG. 1C) increased lifespans and (FIG. 1D) improved motor performance. Note: p<0.0001 for (FIG. 1C) log-rank test; *, **, ***, p<0.05, p<0.01, and p<0.001 respectively, Kruskal-Wallis test (FIG. 1D). (FIG. 1E) Western blots and quantified results of blots depicting greatly reduced SMN in SMA-Mod mutants. **, ***, p<0.01 and p<0.001 respectively, t tests. (FIG. 1F) Diagrammatic representation of the strategy employed to map the SMA modifier to Chr.9. (FIG. 1G) Kaplan-Meier survival curves demonstrating that mutants harboring the C57BL/6-derived Chr.9 ROI are more mildly affected than those homozygous FVB/N for this genomic region. p<0.0001 between SMA-hom C57BL/6 and SMA-hom FVB/N mutants, log-rank test. SMA-hom C57BL/6 Chr.9 mutants were also less severely affected based on (FIG. 1H) the righting reflex assay and (FIG. 1I) their tendency to gain more weight than their SMA-hom FVB/N Chr.9 counterparts. Note: *, **, p<0.05 and p<0.01, Kruskal-Wallis test for panel (H); ***, p<0.001 between SMA-hom C57BL/6 and SMA-hom FVB/N mutants in (I). N.S., not significant. SMA-mod—SMN2^{+/+}; SMNΔ7^{+/+}; Hspa8^{G470R}; Smn^{-/-}, controls—SMN2^{+/+}; SMNΔ7^{+/+}; Smn^{+/-}. Data: mean±SEM.

FIGS. 2A-2E. GWAS links an Hspa8 variant to SMA disease suppression (FIGS. 2A and 2B) Graphical representations of (FIG. 2A) the total number of variant SNPs in the modified SMA mice, and (FIG. 2B) those in protein-coding genes. (FIG. 2C) Pie chart of SNPs filtered for p values<5×10⁸ and predicted for mutational consequences by ANNOVAR. (FIG. 2D) Manhattan plot of highly significant variant SNPs linked to the modified SMA phenotype, emphasizing those clustered in the Chr. 9 ROI; line signifies p=5×10⁸. (FIG. 2E) Restricted view of the Manhattan Plot depicting the Chr. 9 Hspa8 c.G1408C SNP (id: 9:40804249, exon 7) underlying the Hspa8^{G470R} variant (p=1.5×10⁻¹⁵).

FIGS. 3A-3J. Editing of WT Hspa8 to the G470R variant is sufficient to protect against severe SMA. (FIGS. 3A-3D) (A) Sequence chromatograms highlighting the ancestral (GGG) and edited (CGG) codons in Hspa8 from severe and modified SMA mutants. Hspa8^{G470R}-expressing mutants are (FIG. 3B) larger, (FIG. 3C) perform significantly better in a motor performance assay, and (FIG. 3D) have markedly longer lifespans than do mutants expressing the WT protein. Note: *, **, ***, p<0.05, p<0.01, p<0.001, respectively; t tests (SMA and SMA-G470R^{+/+}), one-way ANOVA and log-rank test respectively for (FIG. 3B), (FIG. 3C), and (FIG. 3D). (FIG. 3E) Representative immunostains of lumbar spinal cord sections depicting normal numbers of motor neurons in the SMA-G470R mutant at PND9. (FIG. 3F) Morphometric counts of lumbar motor neurons in SMA and littermate controls. (FIG. 3G) H&E-stained muscle sections from PND9 SMA mutants with or without the G470R variant and a littermate control; myofibers in the SMA-G470R^{+/+} mouse are larger than those of the SMA mutant. (FIGS. 3H and 3I) (FIG. 3H) Frequency distributions and (FIG. 3I) average sizes of myofiber in PND9 SMA and littermate controls. (FIG. 3J) Graph of mean myofiber areas in young adult (PND75) SMA-G470R^{+/+} mutants and littermate controls. Note: *, **, ***, p<0.05, p<0.01, p<0.001, respectively; one-way ANOVA for comparisons at PND9 and t tests for comparisons at PND75 in (FIG. 3F), (FIG. 3I), and (FIG. 3J). N.S., not significant. Scale bars: 20 mm (FIG. 3E) and 25 mm (FIG. 3G). Data: mean±SEM.

FIGS. 4A-4F. NMJ defects in SMA mice are suppressed by the Hspa8^{G470R} variant. (FIG. 4A) Immunostains of NMJs in the triceps of PND9 controls and SMA mutants with or without Hspa8^{G470R}; the modifier reduces denervation (asterisks) and the incidence of nerve terminals with abnormal neurofilament (NF) varicosities (arrows). Scale bars, 20 mm. (FIGS. 4B and 4C) Enumeration of NMJs in the three cohorts of mice displaying (FIG. 4B) nerve terminals abnormally swollen with NF protein and (FIG. 4C) denervated endplates. (FIG. 4D) Graphs depict relative enlargement of endplates in SMA-G470R^{+/+} versus SMA mutants. Note: **, ***, p<0.01, p<0.001, one-way ANOVA (FIGS. 4B-4D). Electrophysiological measures from extensor digitorum longus (EDL) muscles of (FIG. 4E) PND75 SMA-G470R^{+/+} mutants and controls and (FIG. 4F) similarly aged controls with or without the variant illustrate the potentiating effect of the modifier on neurotransmission. Note: *, ***, p<0.05, p<0.001, t tests, N.S., not significant. Data: mean±SEM.

FIGS. 5A-5E. Hspa8^{G470R} alters SMN2 splicing and modestly raises SMN levels (FIGS. 5A and 5B) (FIG. 5A) Western blots of SMN protein in PND9 controls and SMA mutants with or without the G470R modifier and (FIG. 5B) quantified results of the blots in the three cohorts of mice. (FIG. 5C) Analysis of FL-SMN transcript levels by Q-PCR at PND9 in the three cohorts of mice. (FIGS. 5D and 5E) Two-way comparisons of (FIG. 5D) FL-SMN transcript levels and (FIG. 5E) the SMNΔ7 isoform in the two sets of SMA mutants illustrate an increase in the intact transcript and a corresponding drop in the truncated form. Note: *, **, ***, p<0.05, p<0.01, p<0.001, respectively, one-way ANOVA (FIGS. 5B and 5C), t tests, panels (FIGS. 5D and 5E). Data: mean±SEM.

FIGS. 6A-6F. An enhanced affinity of Hspa8^{G470R} for synaptic co-chaperone proteins. (FIG. 6A) Western blot analysis depicting equivalent levels of Hspa8 and other constituent members of a synaptic chaperone complex in PND9 brain tissue of controls and SMA mutants expressing WT or the G470R Hspa8 variant. (FIG. 6B) Quantified results of blot; N.S.—not significant, one-way ANOVA. (FIG. 6C) Co-immunoprecipitation (coIP) analysis of relative affinities of WT Hspa8 or the G470R variant for its co-chaperones, SGTA and CSPα; the variant binds better to SGTA and CSPα. (FIG. 6D) Graph depicting the affinities of Hspa8^{WT} and Hspa8^{G470R} for their interacting partners. (FIG. 6E) Reciprocal coIP analysis of brain-derived Hspa8 and SMN illustrates that the two interact and that there is weakened affinity of the G470R variant for SMN. (FIG. 6F) Quantification of relative affinities of WT Hspa8 or the G470R variant for SMN. Note: *, ***, p<0.05, p<0.001, respectively, t tests for analyses in (FIG. 6D) and (FIG. 6F); brain lysates from PND9 mice were used for coIP experiments. Data: mean±SEM.

FIGS. 7A-7K. SNARE complex assembly is disrupted in SMA NMJs and restored by the Hspa8^{G470R} variant. (FIG. 7A) Representative immunoblot, probed for SNAP25, illustrating reduced high molecular weight SDS-resistant SNARE complexes in PND9 SMA NMJs derived from triceps; complex levels are restored in SMA-G470R mutants. Note: samples were not boiled. (FIG. 7B) Quantified SNARE complex levels in triceps and gastrocnemius muscles of PND9 controls and SMA mutants with or without the G470R variant. Note: **, ***, p<0.01, p<0.001 respectively, one-way ANOVA. N.S.-not significant. (FIG. 7C) Representative immunoblot depicting reduced SNARE complexes in two iPSC-derived motor neuron lines from severe SMA patients; samples were not boiled. (FIG. 7D) Western blot analysis of boiled samples from (FIG. 7C) confirm low levels of SMN in the SMA lines. (FIG. 7E) Graph showing relative SNARE complex concentrations and SMN levels in (FIG. 7C) and (FIG. 7D), respectively. Note: *, **, p<0.05, p<0.01 respectively; one-way ANOVA. (FIG. 7F) Reduced SNARE complex assembly is observed in the immunoblot of samples from PC-12 cells expressing shRNAs against SMN; samples were not boiled. (FIG. 7G) Western blot analysis of boiled samples from (FIG. 7F) confirm low levels of SMN in shRNA-mediated knockdown lines. (FIG. 7H) Quantified SNARE complex and SMN levels in samples analyzed for study depicted in (FIG. 7F) and (FIG. 7G). Note: ***, p<0.001, respectively, t tests for analysis of data in (FIG. 7H). (FIG. 7I) Immunoblot depicting effect of raising Hspa8^{G470R} levels (from a plasmid) on SNARE complex formation in HEK293 cells transfected with the core SNARE components; samples were not boiled. (FIG. 7J) Western blot analysis of boiled samples from (FIG. 7I) showing relatively stable levels of SMN notwithstanding increasing concentrations of Myc-Hspa8^{G470R}, as detected with anti-myc antibody. (FIG. 7K) Quantified levels of SNARE complexes and SMN respectively in (I) and (J). Note: *, **, p<0.05, p<0.01, respectively, one-way ANOVA. N.S.—not significant. Data: mean±SEM.

### DETAILED DESCRIPTION

The present disclosure provides methods and compositions for treating neurodegenerative diseases, including tau-related diseases or tauopathies. Also encompassed by the present disclosure are methods and compositions for potentiating or enhancing neurotransmission. A variant or mutant of a heat shock protein, such as an Hsp70 family member protein, may be used in the present method. Alternatively, a modulator of a heat shock protein may be used.

The Hsp70 family member proteins include, but are not limited to, Hspa8 (Hsp70-8, or Hsc70), Hsp70, Hsp70-2, Hsp70-4, Hsp70-4L, Hsp70-5, Hsp70-6, Hsp70-7, Hsp70-9, Hsp70-12a, and Hsp70-14.

The present method of treating a neurodegenerative disease in a subject may comprise administering an effective amount of a nucleic acid molecule encoding a variant, mutant or modulator of an Hsp70 family member protein (e.g., Hspa8) to the subject. The present method may comprise administering an effective amount of a variant, mutant or modulator of an Hsp70 family member protein (e.g., Hspa8) to the subject.

In one embodiment, the variant of the Hsp70 family member protein is Hspa8^{G470R}

The mutant Hsp70 family member protein (e.g., Hspa8) may have a point mutation, a missense mutation, a nonsense mutation compared to the wildtype Hsp70 family member protein (e.g., Hspa8). The mutation may decrease/disrupt the chaperone activity of the protein (e.g., shifting the function of the protein toward microautophagy such as synaptic microautophagy). The mutation may be in the substrate binding domain of the heat shock protein. The mutation may be in the ATPase domain of the heat shock protein.

The variant or mutant of an Hsp70 family member protein (e.g., Hspa8) may have a lower chaperone activity than the wildtype Hsp70 family member protein (e.g., Hspa8). The variant or mutant of an Hsp70 family member protein (e.g., Hspa8) may have a greater microautophagy activity than the wildtype Hsp70 family member protein (e.g., Hspa8).

The nucleic acid molecule encoding the variant, mutant or modulator of an Hsp70family member protein (e.g., Hspa8) may comprise a viral vector, e.g., a recombinant adeno-associated virus (AAV) vector, encoding the variant, mutant or modulator of an Hsp70 family member protein (e.g., Hspa8).

The present method of treating a neurodegenerative disease in a subject may comprise administering an effective amount of a modulator of an Hsp70 family member protein (e.g., Hspa8) to the subject.

The present method of treating a neurodegenerative disease in a subject may comprise administering an effective amount of a nucleic acid molecule encoding a modulator of an Hsp70 family member protein (e.g., Hspa8) to the subject.

In one embodiment, the modulator binds to a substrate binding domain of an Hsp70 family member protein (e.g., Hspa8). In another embodiment, the modulator binds to an ATPase domain of an Hsp70 family member protein (e.g., Hspa8).

The modulator may decrease a chaperone activity of an Hsp70 family member protein (e.g., Hspa8). The modulator may increase a microautophagy activity of an Hsp70 family member protein (e.g., Hspa8). In one embodiment, the modulator is an inhibitor of an Hsp70 family member protein (e.g., Hspa8).

The modulator may be a small molecule, a polynucleotide (e.g., a small interfering RNA (siRNA) or an antisense molecule), or an antibody or antigen-binding portion thereof. The modulator may comprise a CRISPR/Cas9 system.

The nucleic acid molecule encoding the modulator of an Hsp70 family member protein (e.g., Hspa8) may comprise a viral vector, e.g., a recombinant adeno-associated virus (AAV) vector, encoding the modulator of an Hsp70 family member protein (e.g., Hspa8).

The present composition and method may result in a decrease in the chaperone activity of an Hsp70 family member protein (e.g., Hspa8), where the chaperone activity of an Hsp70 family member protein (e.g., Hspa8) affected by the present composition and method is no greater than 90%, no greater than 85%, no greater than 80%, no greater than 75%, no greater than 70%, no greater than 65%, no greater than 60%, no greater than 55%, no greater than 50%, no greater than 45%, no greater than 40%, no greater than 35%, no greater than 30%, no greater than 25%, no greater than 20%, no greater than 15%, no greater than 10%, about 10% to about 90%, about 15% to about 80%, about 20% to about 70%, about 25% to about 60%, about 30% to about 50%, about 30% to about 40%, about 25% to about 40%, about 20% to about 30%, about 25% to about 35%, about 10% to about 30%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 20% to about 50%, about 12.5% to about 80%, about 20% to about 70%, about 25% to about 60%, or about 25% to about 50%, about 1% to about 100%, about 5% to about 90%, about 10% to about 80%, about 5% to about 70%, about 5% to about 60%, about 10% to about 50%, about 15% to about 40%, about 5% to about 20%, about 1% to about 20%, about 10% to about 30%, about 5%, about 10%, about 20%, about 30%, about 40%, about 50%, about 60%, about 70%, about 80%, about 90%, about 100%, about 10% to about 90%, about 12.5% to about 80%, about 20% to about 70%, about 25% to about 60%, or about 25% to about 50%, of the original chaperone activity of an Hsp70 family member protein (e.g., Hspa8) (in the absence of the present composition and method).

The present composition and method may result in an increase in the autophagy (e.g., microautophagy) activity of an Hsp70 family member protein (e.g., Hspa8), where the autophagy (e.g., microautophagy) activity of an Hsp70 family member protein (e.g., Hspa8) affected by the present composition and method is at least or about 2-fold, at least or about 3-fold, at least or about 4-fold, at least or about 5-fold, at least or about 6-fold, at least or about 7-fold, at least or about 8-fold, at least or about 9-fold, at least or about 10-fold, at least or about 1.1-fold, at least or about 1.2-fold, at least or about 1.3-fold, at least or about 1.4-fold, at least or about 1.5-fold, at least or about 1.6-fold, at least or about 1.8-fold, at least or about 15-fold, at least or about 20-fold, at least or about 50-fold, at least or about 100-fold, at least or about 120-fold, from about 2-fold to about 500-fold, from about 1.1-fold to about 10-fold, from about 1.1-fold to about 5-fold, from about 1.5-fold to about 5-fold, from about 2-fold to about 5-fold, from about 3-fold to about 4-fold, from about 5-fold to about 10-fold, from about 5-fold to about 200-fold, from about 10-fold to about 150-fold, from about 10-fold to about 20-fold, from about 20-fold to about 150-fold, from about 20-fold to about 50-fold, from about 30-fold to about 150-fold, from about 50-fold to about 100-fold, from about 70-fold to about-150 fold, from about 100-fold to about 150-fold, from about 10-fold to about 100-fold, from about 100-fold to about 200-fold, of the original autophagy (e.g., microautophagy) activity of an Hsp70 family member protein (e.g., Hspa8) (in the absence of the present composition and method).

The present composition and method may result in a decrease in the neurodegeneration, the degeneration of neurons, the loss of neurons, neuronal cell death, morphological abnormalities of the neuromuscular junctions (NMJs), etc. of the subject, where the neurodegeneration, the degeneration of neurons, the loss of neurons, neuronal cell death, morphological abnormalities of the neuromuscular junctions (NMJs), etc. of the subject affected by the present composition and method is no greater than 90%, no greater than 85%, no greater than 80%, no greater than 75%, no greater than 70%, no greater than 65%, no greater than 60%, no greater than 55%, no greater than 50%, no greater than 45%, no greater than 40%, no greater than 35%, no greater than 30%, no greater than 25%, no greater than 20%, no greater than 15%, no greater than 10%, about 10% to about 90%, about 15% to about 80%, about 20% to about 70%, about 25% to about 60%, about 30% to about 50%, about 30% to about 40%, about 25% to about 40%, about 20% to about 30%, about 25% to about 35%, about 10% to about 30%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 20% to about 50%, about 12.5% to about 80%, about 20% to about 70%, about 25% to about 60%, or about 25% to about 50%, about 1% to about 100%, about 5% to about 90%, about 10% to about 80%, about 5% to about 70%, about 5% to about 60%, about 10% to about 50%, about 15% to about 40%, about 5% to about 20%, about 1% to about 20%, about 10% to about 30%, about 5%, about 10%, about 20%, about 30%, about 40%, about 50%, about 60%, about 70%, about 80%, about 90%, about 100%, about 10% to about 90%, about 12.5% to about 80%, about 20% to about 70%, about 25% to about 60%, or about 25% to about 50%, of the neurodegeneration, the degeneration of neurons, the loss of neurons, neuronal cell death, morphological abnormalities of the neuromuscular junctions (NMJs), etc. of the subject in the absence of the present composition and method.

The present method and composition may ameliorate the symptoms of a neurodegenerative disease or disorder in a subject. The present method and composition may result in at least partial correction of neuropathology, and/or alleviation and/or prevention and/or stabilization and/or slowing of disease progression, and/or progression of the symptoms of a neurodegenerative disease or disorder. The present method and composition may prevent neuron death, and/or delay the onset of paralysis and death.

The present composition and method may result in an increase in motor neuron number, neuromuscular junction (NMJ) electrophysiology (e.g., miniature end-plate potentials (mEPPs), end-plate potentials (EPPs), Quantal content), neurotransmission at the NMJ, muscle strength, etc. of the subject, where the motor neuron number, neuromuscular junction (NMJ) electrophysiology (e.g., miniature end-plate potentials (mEPPs), end-plate potentials (EPPs), Quantal content), neurotransmission at the NMJ, muscle strength, etc. of the subject affected by the present composition and method is at least or about 2-fold, at least or about 3-fold, at least or about 4-fold, at least or about 5-fold, at least or about 6-fold, at least or about 7-fold, at least or about 8-fold, at least or about 9-fold, at least or about 10-fold, at least or about 1.1-fold, at least or about 1.2-fold, at least or about 1.3-fold, at least or about 1.4-fold, at least or about 1.5-fold, at least or about 1.6-fold, at least or about 1.8-fold, at least or about 15-fold, at least or about 20-fold, at least or about 50-fold, at least or about 100-fold, at least or about 120-fold, from about 2-fold to about 500-fold, from about 1.1-fold to about 10-fold, from about 1.1-fold to about 5-fold, from about 1.5-fold to about 5-fold, from about 2-fold to about 5-fold, from about 3-fold to about 4-fold, from about 5-fold to about 10-fold, from about 5-fold to about 200-fold, from about 10-fold to about 150-fold, from about 10-fold to about 20-fold, from about 20-fold to about 150-fold, from about 20-fold to about 50-fold, from about 30-fold to about 150-fold, from about 50-fold to about 100-fold, from about 70-fold to about-150 fold, from about 100-fold to about 150-fold, from about 10-fold to about 100-fold, from about 100-fold to about 200-fold, of the motor neuron number, neuromuscular junction (NMJ) electrophysiology (e.g., miniature end-plate potentials (mEPPs), end-plate potentials (EPPs), Quantal content), neurotransmission at the NMJ, muscle strength, etc. of the subject in the absence of the present composition and method.

The present pharmaceutical composition may comprise, or consist essentially of (or consist of), a nucleic acid molecule encoding a variant, mutant or modulator of an Hsp70 family member protein (e.g., Hspa8).

The present pharmaceutical composition may comprise, or consist essentially of (or consist of), a variant, mutant or modulator of an Hsp70 family member protein (e.g., Hspa8).

The present pharmaceutical composition (the nucleic acid molecule encoding the variant, mutant or modulator of an Hsp70 family member protein (e.g., Hspa8), the variant, mutant or modulator of an Hsp70 family member protein (e.g., Hspa8)) may be administered to the central nervous system (CNS) of the subject. The present pharmaceutical composition may be administered to the spinal cord or brain (e.g., the brainstem region) of the subject. The present pharmaceutical composition may be administered by intrathecal, intraventricular (known also as intracerebroventricular or ICV), intracranial, or intramuscular administration (e.g., injection). The present pharmaceutical composition may be administered to a particular ventricle, e.g., to the lateral ventricles or to the fourth ventricle of the brain. The present pharmaceutical composition may be administered by stereotaxic microinjection.

In one embodiment, the present pharmaceutical composition may be administered via a pump. Such pumps are commercially available, for example, from Alzet (Cupertino, Calif.) or Medtronic (Minneapolis, Minn.). The pump may be implantable. Another way to administer the present pharmaceutical composition is to use a cannula or a catheter.

The present pharmaceutical composition may be administered intrathecally, orally, intravenously, intramuscularly, topically, arterially, or subcutaneously.

The present composition and methods may be used to treat a neurodegenerative disorder. Non-limiting examples of neurodegenerative disorder include, amyotrophic lateral sclerosis, multiple sclerosis, Parkinson's disease, Alzheimer's disease, Huntington's disease, multiple system atrophy, Batten disease, or a prion disease.

The present composition and methods may be used to treat a condition with defects in neurotransmission stemming from perturbed synaptic autophagy.

In certain embodiments, the present composition and methods may enhance neurotransmission, with or without a significant effect on SMN levels. In certain embodiments, the present composition and methods may increase SMN levels.

The present composition and methods may also be used to treat conditions including, but not limited to, spinal cerebellar ataxia, spinal muscular atrophy, traumatic spinal cord injury, and Tay-Sachs disease.

The present method may further comprise administering an SMN2 splicing modifier to the subject.

The heat shock protein may be an Hsp70 family member protein, including, but not limited to, Hspa8 (Hsp70-8, Hsc70), Hsp70, Hsp70-2, Hsp70-4, Hsp70-4L, Hsp70-5, Hsp70-6, Hsp70-7, Hsp70-9, Hsp70-12a, and Hsp70-14.

Heat shock 70 kDa protein 8 (Hspa8), also known as Hsp70-8 or Hsc70, is a heat shock protein that in humans is encoded by the HSPA8 gene on chromosome 11 (Gene ID 3312). The murine HSPA8 has a Gene ID of 15481. As a member of the heat shock protein 70 family and a chaperone protein, it facilitates the proper folding of newly translated and misfolded proteins, as well as stabilize or degrade mutant proteins. Its functions contribute to biological processes including signal transduction, apoptosis, autophagy, protein homcostasis, and cell growth and differentiation. Mayer et al., Hsp70 chaperones: cellular functions and molecular mechanism, Cellular and Molecular Life Sciences, 2005, 62 (6): 670-684. The Hsp70 proteins have three major functional domains: an N-terminal ATPase domain, a substrate binding domain, and a C-terminal domain.

The NCBI Reference Sequence (RefSeq) accession numbers for human Hspa8 mRNA may include NM_006597 and NM_153201. The NCBI RefSeq accession numbers for human Hspa8 protein may include NP_006588 and NP_694881. The NCBI RefSeq accession numbers for murine Hspa8 mRNA may include NM_031165 and NM_001364480. The NCBI RefSeq accession numbers for murine Hspa8 protein may include NP_112442 and NP_001351409.

There may be a number of different isoforms for each of these heat shock proteins discussed in this disclosure, provided herein are the general accession numbers, NCBI Reference Sequence (RefSeq) accession numbers, GenBank accession numbers, and/or UniProt numbers to provide relevant sequences. The proteins/polypeptides may also comprise other sequences. Any isoform and transcript variants of the Hsp70 family member protein (e.g., Hspa8) are encompassed by the present disclosure.

As used herein, the terms “variant” and “mutant” may refer to an Hsp70 family member protein (e.g., Hspa8) having an amino acid sequence that differs in some respect from a standard or reference sequence (e.g., in some embodiments, a wildtype sequence). The difference may be referred to as a “mutation”. In some embodiments, a mutant is a polypeptide sequence that has been altered by at least one substitution, insertion, cross-over, deletion, and/or other genetic operation. Mutants and variants are not limited to a particular method by which they are generated. In some embodiments, a mutant or variant sequence has increased, decreased, or substantially similar activities or properties, in comparison to the wildtype sequence. In some embodiments, the variant comprises one or more amino acid residues that have been mutated, as compared to the amino acid sequence of the wild-type polypeptide. The term “variant” may refer to any of several different forms of an Hsp70 family member protein (e.g., Hspa8). Variants may arise due to alternative splicing of mRNA encoding the protein, post-translational modification of the protein, proteolytic processing of the protein, genetic variations and/or somatic recombination.

The present modulator may modulate the activity and/or level of any isoform of the heat shock protein (e.g., an Hsp70 family member protein such as Hspa8). The present modulator may modulate the activity and/or level of a wild-type, variant or mutant heat shock protein (e.g., an Hsp70 family member protein such as Hspa8).

The term “Hspa8”, “Hspa8” “HSPA8” or “HSPA8” is meant to include the DNA, RNA, mRNA, cDNA, recombinant DNA or RNA, or the protein encoded by the gene. As used herein, Hspa8 can refer to the gene or the protein encoded by the gene, as appropriate in the specific context utilized. Additionally, in certain contexts, the reference will be to the mouse gene or protein, and in others the human gene or protein as appropriate in the specific context.

The present composition and methods may be used in combination with other therapeutic treatments for the condition, such as an agent that increases the level of SMN protein. For example, the agent may be an SMN2 splicing modifier. The SMN2 splicing modifier may act by shifting SMN2 pre-mRNA splicing toward the production of full length SMN mRNA. The SMN2 splicing modifier may modulate alternate splicing of the survival motor neuron 2 (SMN2) gene, functionally converting it into SMN1 gene, thus increasing the level of SMN protein in the CNS (e.g., Spinraza®). The other therapeutic treatments may inhibit glutamate release (e.g., riluzole). The other therapeutic treatments may be a metalloporphyrin that neutralizes reactive oxygen and nitrogen species. The other therapeutic treatments may be a JNK Inhibitor, or an antioxidant that scavenges reactive oxygen species (ROS) and inhibits proinflammatory responses.

The routes of administration of the pharmaceutical compositions include oral, intravenous, subcutaneous, intramuscular, inhalation, or intranasal administration. Additionally, specifically targeted delivery of the present composition (comprising, e.g., nucleic acid, protein/polypeptide, or small molecule) could be delivered by targeted liposome, nanoparticle or other suitable means.

The amount and/or activity of an Hsp70 family member protein (e.g., Hspa8) may be downregulated by RNA interference or RNAi (such as small interfering RNAs or siRNAs, small hairpin RNAs or shRNAs, microRNAs or miRNAs, a double-stranded RNA (dsRNA), etc.), antisense molecules, and/or ribozymes targeting the DNA or mRNA encoding the Hsp70 family member protein (e.g., Hspa8). The amount and/or activity of an Hsp70 family member protein (e.g., Hspa8) may be downregulated by gene knockout. The amount and/or activity of an Hsp70 family member protein (e.g., Hspa8) may be downregulated by the cluster regularly interspaced short palindromic repeat-associated nuclease (CRISPR) technology.

The amount and/or activity of an Hsp70 family member protein (e.g., Hspa8) may be modulated by introducing polypeptides (e.g., antibodies) or small molecules which inhibit gene expression or functional activity of an Hsp70 family member protein (e.g., Hspa8). Agents that bind to or modulate, such as downregulating the amount, activity or expression of an Hsp70 family member protein (e.g., Hspa8), may be administered to a subject or target cells. Such an agent may be administered in an amount effective to downregulate the expression and/or activity of an Hsp70 family member protein (e.g., Hspa8), or by activating or downregulating a second signal which controls the expression, activity or amount of the Hsp70 family member protein (e.g., Hspa8).

Methods and compositions of the present disclosure may be used for prophylaxis as well as treating a disease as described herein (such as a neurodegenerative disease).

For prophylaxis, the present composition may be administered to a subject in order to prevent the onset of one or more symptoms of a neurodegenerative disease. In one embodiment, the subject is asymptomatic. A prophylactically effective amount of the agent or composition is administered to such a subject. A prophylactically effective amount is an amount which prevents the onset of one or more symptoms of the neurodegenerative disease.

The present compositions may be used in vitro or administered to a subject. The administration may be topical, intravenous, intranasal, or any other suitable route as described herein.

The present methods may utilize adeno-associated virus (AAV) mediated gene delivery. Additionally, delivery vehicles such as nanoparticle-and lipid-based nucleic acid or protein delivery systems can be used as an alternative to viral vectors. Further examples of alternative delivery vehicles include lentiviral vectors, lipid-based delivery system, gene gun, hydrodynamic, electroporation or nucleofection microinjection, and biolistics. Various gene delivery methods are discussed in detail by Nayerossadat et al. (Adv Biomed Res. 2012; 1:27) and Ibraheem et al. (Int J Pharm. 2014 Jan. 1; 459(1-2): 70-83).

The present methods may use nanoparticle-based siRNA delivery systems. The nanoparticle-formulated siRNA delivery systems may be based on polymers or liposomes. Nanoparticles conjugated to the cell-specific targeting ligand for effective siRNA delivery can increase the chance of binding the cell surface receptor. The nanoparticles may be coated with PEG (polyethylene glycol) which can reduce uptake by the reticuloendothelial system (RES), resulting in enhanced circulatory half-life. Various nanoparticle-based delivery systems such as cationic lipids, polymers, dendrimers, and inorganic nanoparticles may be used in the present methods to provide effective and efficient siRNA delivery in vitro or in vivo.

The present composition may be administered by bolus injection or chronic infusion. The present composition may be administered directly into the central nervous system (CNS). The present composition may be administered systemically. The present composition may be administered by topical administration, intraocular administration, parenteral administration, intrathecal administration, subdural administration, and/or subcutaneous administration. The present composition may be administered at or near the site of the disease, disorder or injury, in an effective amount.

The present composition may be administered in a local or systemic manner, for example, via injection directly into the desired target site, e.g., in a depot or sustained release formulation. The composition may be administered in a targeted drug delivery system, for example, in liposomes or nanoparticles coated with tissue-specific or cell-specific ligands/antibodies. The liposomes or nanoparticles will be targeted to and taken up selectively by the desired tissue or cells. A summary of various delivery methods and techniques of siRNA administration in ongoing clinical trials is provided in Zuckerman and Davis 2015; Nature Rev. Drug Discovery, Vol. 14:843-856, December 2015. In some embodiments, the level of an Hsp70 family member protein (e.g., Hspa8) is decreased in a desired target cell. The expression of the Hsp70 family member protein (e.g., Hspa8) may be specifically decreased only in the desired target cell (i.e., those cells which are predisposed to the condition, or exhibiting the disease already), and not substantially in other non-diseased cells. In these methods, expression of the Hsp70 family member protein (e.g., Hspa8) may not be substantially reduced in other cells, i.e., cells which are not desired target cells. Thus, in such embodiments, the level of the Hsp70 family member protein (e.g., Hspa8) remains substantially the same or similar in non-target cells in the course of or following treatment.

The vectors comprising the present nucleic acid may be delivered into host cells by a suitable method. Methods of delivering the present composition to cells may include transfection of nucleic acids or polynucleotides (e.g., using reagents such as liposomes or nanoparticles); electroporation, delivery of protein, e.g., by mechanical deformation (see, e.g., Sharci et al. Proc. Natl. Acad. Sci. USA (2013) 110 (6): 2082-2087); or viral transduction. Exemplary viral vectors include, but are not limited to, recombinant retroviruses, alphavirus-based vectors, and adeno-associated virus (AAV) vectors. In some embodiments, the vectors are retroviruses. In some embodiments, the vectors are lentiviruses. In some embodiments, the vectors are adeno-associated viruses.

Vectors of the present disclosure can comprise any of a number of promoters known to the art, wherein the promoter is constitutive, regulatable or inducible, cell type specific, tissue-specific, or species specific. In addition to the sequence sufficient to direct transcription, a promoter sequence of the invention can also include sequences of other regulatory elements that are involved in modulating transcription (e.g., enhancers, kozak sequences and introns). Many promoter/regulatory sequences useful for driving constitutive expression of a gene are available in the art and include, but are not limited to, for example, CMV (cytomegalovirus promoter), EF1a (human elongation factor 1 alpha promoter), SV40 (simian vacuolating virus 40 promoter), PGK (mammalian phosphoglycerate kinase promoter), Ubc (human ubiquitin C promoter), human beta-actin promoter, rodent beta-actin promoter, CBh (chicken beta-actin promoter), CAG (hybrid promoter contains CMV enhancer, chicken beta actin promoter, and rabbit beta-globin splice acceptor), TRE (Tetracycline response element promoter), H1 (human polymerase III RNA promoter), U6 (human U6 small nuclear promoter), and the like. Moreover, inducible and tissue specific expression of an RNA, transmembrane proteins, or other proteins can be accomplished by placing the nucleic acid encoding such a molecule under the control of an inducible or tissue specific promoter/regulatory sequence. Examples of tissue specific or inducible promoter/regulatory sequences which are useful for this purpose include, but are not limited to, the rhodopsin promoter, the MMTV LTR inducible promoter, the SV40 late enhancer/promoter, synapsin 1 promoter, ET hepatocyte promoter, GS glutamine synthase promoter and many others. Various commercially available ubiquitous as well as tissue-specific promoters can be found at http://www.invivogen.com/prom-a-list and https://www.addgene.org/. In addition, promoters which are well known in the art can be induced in response to inducing agents such as metals, glucocorticoids, tetracycline, hormones, and the like, are also contemplated for use with the invention. Thus, it will be appreciated that the present disclosure includes the use of any promoter/regulatory sequence known in the art that is capable of driving expression of the desired protein operably linked thereto.

Vectors according to the present disclosure can be transformed, transfected or otherwise introduced into a wide variety of host cells. Numerous methods of transfection are known to the ordinarily skilled artisan, for example, lipofectamine, calcium phosphate co-precipitation, electroporation, DEAE-dextran treatment, microinjection, viral transduction, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

The administration regimen may depend on several factors, including the serum or tissue turnover rate of the therapeutic composition, the level of symptoms, and the accessibility of the target cells in the biological matrix. Preferably, the administration regimen delivers sufficient therapeutic composition to effect improvement in the target disease state, while simultaneously minimizing undesired side effects.

In accordance with the present disclosure, there may be numerous tools and techniques within the skill of the art, such as those commonly used in molecular immunology, cellular immunology, pharmacology, and microbiology (sec, e.g., Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.)

As used herein, the term “modulator” refers to agents capable of modulating (e.g., down-regulating, decreasing, suppressing, or upregulating, increasing) the level/amount and/or activity of the heat shock protein (e.g., Hspa8).

As used herein, the term “inhibitor” refers to agents capable of down-regulating or otherwise decreasing or suppressing the level/amount and/or activity of the heat shock protein (e.g., Hspa8).

The mechanism of modulation may be at the genetic level (e.g., modulating such as interfering with, inhibiting, down-regulating, decreasing, suppressing, or upregulating, increasing, expression, transcription or translation, etc.) or at the protein level (e.g., binding, competition, etc.).

The mechanism of inhibition may be at the genetic level (e.g., interference with or inhibit expression, transcription or translation, etc.) or at the protein level (e.g., binding, competition, etc.).

The present modulators may be a small molecule, a polynucleotide, a polypeptide, or an antibody or antigen-binding portion thereof. In one embodiment, the polynucleotide is a small interfering RNA (siRNA) or an antisense molecule.

In one embodiment, the modulator is a CRISPR (clustered regularly interspaced short palindromic repeats)-Cas system specific for the heat shock protein (e.g., Hspa8).

A wide variety of suitable modulators may be employed, guided by art-recognized criteria such as efficacy, toxicity, stability, specificity, half-life, etc.

### Modulators of Heat Shock Proteins

### Small Molecule Modulators

As used herein, the term “small molecules” encompasses molecules other than proteins or nucleic acids without strict regard to size. Non-limiting examples of small molecules that may be used according to the methods and compositions of the present invention include, small organic molecules, peptide-like molecules, peptidomimetics, carbohydrates, lipids or other organic (carbon containing) or inorganic molecules.

Non-limiting examples of the present modulators of heat shock proteins include sulfogalactolipids (SGLs), sulfogalactosyl ceramide (SGC), and sulfogalactoglycerolipid (SGG). In one embodiment, the SGL, SGC, or SGG bind to the N-terminal ATPase-containing domain of an Hsp70 family member. Mamelak et al., Carbohydrate Research, 2001, 335(2):91-100.

Non-limiting examples of the present modulators of heat shock proteins include the compounds described in U.S. Pat. Nos. 10,052,325; 9,567,318; and U.S. Patent Publication No. 2009-0075948.

Non-limiting examples of the present modulators of autophagy (e.g., microautophagy such as synaptic microautophagy) include the compounds described in WO2017098467, and WO2014026372.

In certain embodiments, the inhibitor used in the present methods and compositions is a polynucleotide that reduces expression of an Hsp70 family member protein (e.g., Hspa8).

The nucleic acid target of the polynucleotides (e.g., siRNA, antisense oligonucleotides, and ribozymes) may be any location within the gene or transcript of an Hsp70 family member protein (e.g., Hspa8).

### RNA Interference

SiRNAs (small interfering RNAs) or small-hairpin RNA (shRNA) may be used to modulate (e.g., decrease) the level of an Hsp70 family member protein (e.g., Hspa8).

SiRNAs may have 16-30 nucleotides, e.g., 16, 17, 18, 19, 20, 21, 22, 23, 24, 25, 26, 27, 28, 29, or 30 nucleotides. The siRNAs may have fewer than 16 or more than 30 nucleotides. The polynucleotides of the invention include both unmodified siRNAs and modified siRNAs such as siRNA derivatives etc.

SiRNAs can be delivered into cells in vitro or in vivo by methods known in the art, including cationic liposome transfection and electroporation. SiRNAs and shRNA molecules can be delivered to cells using viruses or DNA vectors.

### Antisense Polynucleotides

In other embodiments, the polynucleotide is an antisense molecule that is complementary to a target region within the mRNA of an Hsp70 family member protein (e.g., Hspa8). The antisense polynucleotide may bind to the target region and inhibit translation. The antisense oligonucleotide may be DNA or RNA, or comprise synthetic analogs of ribo-deoxynucleotides. Thus, the antisense oligonucleotide inhibits expression of an Hsp70 family member protein (e.g., Hspa8).

An antisense oligonucleotide can be, for example, about 7, 10, 15, 20, 25, 30, 35, 40, 45, 50, 55, 60, 65, 70, 75, 80, or more nucleotides in length.

The antisense nucleic acid molecules of the invention may be administered to a subject, or generated in situ such that they hybridize with or bind to the mRNA of an Hsp70 family member protein (e.g., Hspa8). Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using viruses or DNA vectors.

### Ribozyme In other embodiments, the polynucleotide is a ribozyme that inhibits expression of the gene of an Hsp70 family member protein (e.g., Hspa8).

Ribozymes can be chemically synthesized in the laboratory and structurally modified to increase their stability and catalytic activity using methods known in the art. Alternatively, ribozyme encoding nucleotide sequences can be introduced into host cells through gene-delivery mechanisms known in the art.

Other aspects of the invention include vectors (e.g., viral vectors, expression cassettes, plasmids) comprising or encoding polynucleotides of the subject invention (e.g., siRNA, antisense nucleic acids, and ribozymes), and host cells genetically modified with polynucleotides or vectors of the subject invention.

### Polypeptides

The present modulators can be a polypeptide that modulates the activity and/or level of an Hsp70 family member protein (e.g., Hspa8). The modulator may be an inhibitor which is a polypeptide decreasing/inhibiting the activity and/or level of an Hsp70 family member protein (e.g., Hspa8).

Various means for delivering polypeptides to a cell can be utilized to carry out the methods of the subject invention. For example, protein transduction domains (PTDs) can be fused to the polypeptide, producing a fusion polypeptide, in which the PTDs are capable of transducing the polypeptide cargo across the plasma membrane (Wadia, J. S. and Dowdy, S. F., Curr. Opin. Biotechnol., 2002, 13(1)52-56).

According to the present methods, recombinant cells may be administered to a subject, wherein the recombinant cells have been genetically modified to express a nucleotide sequence encoding a modulatory or inhibitory polypeptide.

### Antibodies

The present modulators can be an antibody or antigen-binding portion thereof that is specific to an Hsp70 family member protein (e.g., Hspa8).

The antibody or antigen-binding portion thereof may be the following: (a) a whole immunoglobulin molecule; (b) an scFv; (c) a Fab fragment; (d) an F(ab′)2; and (e) a disulfide linked Fv. The antibody or antigen-binding portion thereof may be monoclonal, polyclonal, chimeric and humanized. The antibodies may be murine, rabbit or human antibodies.

### Endonucleases

The Hsp70 family member protein (e.g., Hspa8) may be modulated (e.g., inhibited) by using a sequence-specific endonuclease that target the gene encoding the Hsp70 family member protein (e.g., Hspa8). Thus, the modulator (e.g., an inhibitor) of an Hsp70 family member protein (e.g., Hspa8) may comprise an endonuclease.

Non-limiting examples of the endonucleases include a zinc finger nuclease (ZFN), a ZFN dimer, a ZFNickase, a transcription activator-like effector nuclease (TALEN), or an RNA-guided DNA endonuclease (e.g., CRISPR/Cas9). Meganucleases are endonucleases characterized by their capacity to recognize and cut large DNA sequences (12 base pairs or greater). Any suitable meganuclease may be used in the present methods to create double-strand breaks in the host genome, including endonucleases in the LAGLIDADG and PI-Sce family.

An example of sequence-specific endonucleases includes RNA-guided DNA nucleases, e.g., the CRISPR/Cas system (Geurts et al., Science 325, 433 (2009); Mashimo et al., PLOS ONE 5, e8870 (2010); Carbery et al., Genetics 186, 451-459 (2010); Tesson et al., Nat. Biotech. 29, 695-696 (2011). Wiedenheft et al. Nature 482,331-338 (2012); Jinek et al. Science 337,816-821 (2012); Mali et al. Science 339,823-826 (2013); Cong et al. Science 339,819-823 (2013)).

### Conditions to be Treated

The present disclosure relates to a method of treating or preventing a neurodegenerative disease, disorder or condition in a subject.

A neurodegenerative disease may be caused by the progressive loss of structure or function of neurons, in the process known as neurodegeneration. Such neuronal damage may ultimately involve cell death. Neurodegeneration can be found in the brain at many different levels of neuronal circuitry, ranging from molecular to systemic. Neurodegenerative diseases may include abnormalities in signaling pathways, for example aberrant phosphorylation due to dysregulated kinase activity, mutant proteins (mutant tau, mutant APP) and chaperone unbalance leading to misfolding. Neurodegenerative diseases may be characterized by a slow progressive loss of neurons in the central nervous system (CNS), which often leads to deficits in specific brain functions (e.g., memory, movement, cognition, etc.) performed by the affected CNS region.

Neurodegenerative diseases may include amyotrophic lateral sclerosis, multiple sclerosis, Parkinson's disease, Alzheimer's disease, Huntington's disease, multiple system atrophy, Batten disease, and prion diseases such as Creutzfeldt-Jakob disease.

Neurodegenerative diseases include, but are not limited to, Alzheimer's disease (sporadic or familial), amyotrophic lateral sclerosis (ALS), Parkinson's disease (PD), multiple sclerosis, Huntington's disease, multiple system atrophy, argyrophilic grain dementia, dementia pugilistica, chronic traumatic encephalopathy, diffuse neurofibrillary tangles with calcification, Down syndrome, Gerstmann-Straussler-Scheinker disease, Hallervorden-Spatz disease, hereditary frontotemporal dementia, parkinsonism linked to chromosome 17 (FTDP-17), inclusion body myositis, Creutsfeld-Jakob disease, multiple system atrophy, Niemann-Pick disease type C, Pick's disease, prion protein cerebral amyloid angiopathy, sporadic corticobasal degeneration, progressive supranuclear palsy, subacute sclerosing panencephalitis, myotonic dystrophy, motor neuron disease with neurofibrillary tangles, tangle only dementia, and progressive subcortical gliosis. Neurodegenerative diseases also include alcohol-induced neurodegeneration; brain ischemia; cocaine addiction; diffuse Lewy body disease; electroconvulsive seizures; fetal alcohol syndrome; focal cortical dysplasia; hereditary canine spinal muscular atrophy; inclusion body myositis; multiple system atrophy; Niemann-Pick type C; Parkinson's disease; and peripheral nerve injury.

Neurodegenerative diseases or conditions may include Parkinson's disease, Alzheimer's disease, prion disease, a motor neuron disease (MND) such as amyotrophic lateral sclerosis (ALS), Huntington's disease (HD), spinocerebellar ataxia (SCA), spinal muscular atrophy (SMA), Friedreich's ataxia, Lewy body disease, epilepsy, encephalitis, hydrocephalus, stroke, chronic traumatic encephalopathy (CTE); a synucleinopathy; a tauopathy, a spongiform encephalopathy; familial amyloidotic polyneuropathy; Dutch hereditary cerebral hemorrhage with amyloidosis; congophilic angiopathy; corticobasal degeneration; Pick's disease; progressive supranuclear palsy; Creutzfeldt-Jacob disease; Gerstmann-Straussler-Schneiker syndrome; fatal familial insomnia; kuru; bovine spongiform encephalopathy; scrapie; chronic wasting disease; Lewy body variant of Alzheimer's disease; diffuse Lewy body disease; dementia with Lewy bodies; multiple system atrophy; neurodegeneration with brain iron accumulation type I; diffuse Lewy body disease; frontotemporal lobar degeneration; hereditary dentatorubral-pallidoluysian atrophy; Kennedy's disease; Alexander's disease; Cockayne syndrome; Icelandic hereditary cerebral hemorrhage with amyloidosis; and neuroinflammation.

As used herein, the terms “neurodegenerative disease”, “neurodegenerative disorder”, and “neurodegenerative condition” generally refer to any disease, disorder, and/or condition that affects the neurons (sometimes referred to as “nerve cells”), such as neurons of a brain and/or neurons of a nervous system which is associated with the degeneration or loss of neural cells. Neurodegenerative diseases may result in progressive degeneration and/or death of nerve cells. In general neurodegeneration is the progressive loss of structure and/or function of neurons, including the death of neurons. Neurodegenerative diseases may cause problems with movement (e.g., ataxias), or mental or cognitive functioning (e.g., dementias). Frequently neurodegeneration is associated with neuroinflammation. Therefore, it is to be understood that neurodegenerative diseases or disorders encompass neural diseases which are characterized by neuroinflammation. Sometimes in such diseases activated microglia may produce inflammatory cytokines that contribute to widespread inflammation and may lead to and/or result in a neurodegenerative condition and/or disease. Some neurodegenerative diseases and/or conditions are associated with microglia cell over-activation, increased numbers of microglia cells, production of inflammatory proteins and/or inflammatory activities, and/or neuronal death.

Non-limiting examples of neurodegenerative diseases include Alzheimer's disease and other dementias, Parkinson's disease and other Parkinson's disease related disorders, prion disease, motor neuron diseases other than ALS, Huntington's disease, Spinocerebellar ataxia (SCA), Spinal muscular atrophy (SMA), Friedreich's ataxia, Lewy body disease, epilepsy, multiple sclerosis, encephalitis, hydrocephalus, stroke, chronic traumatic encephalopathy (CTE); synucleinopathies; tauopathies; spongiform encephalopathics; familial amyloidotic polyneuropathy; Dutch hereditary cerebral hemorrhage with amyloidosis; congophilic angiopathy; corticobasal degeneration; Pick's disease; progressive supranuclear palsy; Creutzfeld-Jacob disease; Gerstmann-Straussler-Schneiker syndrome; fatal familial insomnia; kuru; bovine spongiform encephalopathy; scrapie; chronic wasting disease; Lewy body variant of Alzheimer's disease; diffuse Lewy body disease; dementia with Lewy bodies; multiple system atrophy; neurodegeneration with brain iron accumulation type I; diffuse Lewy body disease; frontotemporal lobar degeneration; hereditary dentatorubral-pallidoluysian atrophy; Kennedy's disease; Alexander's disease; Cockayne syndrome; Icelandic hereditary cerebral hemorrhage with amyloidosis.

In some embodiments, the present method/composition may decrease or prevent at least one symptom associated with a neurodegenerative disease.

Tau (tubulin associated unit, or MAPT) plays an important role in the morphology and physiology of neurons. A disease associated with tau deposition in the brain may be referred to as a “tauopathy”. A tauopathy may encompass any neurodegenerative disease that involves the pathological aggregation of tau within the brain. A tauopathy may include neurodegenerative diseases characterized by tau protein abnormalities that share the feature of hyperphosphorylated tau protein, and intracellular neurofibrillary tangle (NFT) formation.

Tauopathy may include neurodegenerative diseases involving the aggregation of tau protein into neurofibrillary or gliofibrillary tangles in the human brain. Tangles may be formed by hyperphosphorylation of tau, causing the protein to dissociate from microtubules and form insoluble aggregates. Tauopathies may include neurodegenerative disease where tau occurs in a highly phosphorylated form, detaches from microtubules, and aggregates. Pathogenic tau mutations or abnormal tau phosphorylation result in a more rapid development of NFTs and neurologic disease.

Tauopathies include, but are not limited to, Alzheimer's disease (familial or sporadic), progressive supranuclear palsy, dementia pugilistica, chronic traumatic encephalopathy, frontotemporal dementia and parkinsonism linked to chromosome 17 (FTDP-17), Lytico-Bodig disease, Parkinson-dementia complex of Guam, tangle-predominant dementia, tangle only dementia, ganglioglioma and gangliocytoma, meningioangiomatosis, subacute sclerosing panencephalitis, lead encephalopathy, tuberous sclerosis, Hallervorden-Spatz disease, lipofuscinosis, Pick's disease, progressive supranuclear palsy, progressive subcortical gliosis, corticobasal degeneration, diffuse neurofibrillary tangles with calcification, argyrophilic grain disease (AGD), argyrophilic grain dementia, frontotemporal lobar degeneration, frontotemporal dementia, amyotrophic lateral sclerosis parkinsonism-dementia complex, dementia pugilistica, Down syndrome, Gerstmann-Straussler-Scheinker disease, inclusion body myositis, Creutzfeld-Jakob disease, multiple system atropy, Niemann-Pick disease type C, prion protein cerebral amyloid angiopathy, subacute sclerosing panencephalitis, myotonic dystrophy, non-guanamian motor neuron disease with neurofibrillary tangles, postencephalitic parkinsonism, and chronic traumatic encephalopathy.

Tauopathies include neurodegenerative disorders characterized by neuronal and/or glial taupositive inclusions. Clinically, tauopathies can present with a range of phenotypes that include cognitive/behavioral-disorders, movement disorders, language disorders and non-specific amnestic symptoms in advanced age. Pathologically, tauopathies can be classified based on the predominant tau isoforms that are present in the inclusion bodies (i.e., 3R, 4R or equal 3R: 4R ratio). “Tauopathies” may include neurodegenerative disorders characterized by tau deposits in the brain (mainly in neurons, also in glial cells and extracellular space), with symptoms of dementia and parkinsonism. Depending on the major tau isoforms appearing in aggregates, tauopathies are usually classified into 3R tauopathies (mainly having 3R tau), 4R tauopathies (mainly having 4R tau) and 3R/4R tauopathies (with approximately an equal ratio of 3R tau and 4R tau). Besides, in primary tauopathies, tau is the major and prominent component of the pathology, such as PiD (Pick's disease), PSP (Progressive supranuclear palsy), CBD (corticobasal degeneration) and AGD (Argyrophilic grain disease).

The present method/composition may promote the clearance of tau aggregates from the brain of a subject. The clearance of tau aggregates includes clearance of neurofibrillary tangles and/or the pathological tau precursors to neurofibrillary tangles.

The present method/composition may slow the progression of a tau-pathology related behavioral phenotype in a subject. As used herein, a tau-pathology related behavioral phenotype includes, without limitation, cognitive impairments, early personality change and disinhibition, apathy, abulia, mutism, apraxia, perseveration, stereotyped movements/behaviors, hyperorality, disorganization, inability to plan or organize sequential tasks, selfishness/callousness, antisocial traits, a lack of empathy, halting, agrammatic speech with frequent paraphasic errors but relatively preserved comprehension, impaired comprehension and word-finding deficits, slowly progressive gait instability, retropulsions, freezing, frequent falls, non-levodopa responsive axial rigidity, supranuclear gaze palsy, square wave jerks, slow vertical saccades, pseudobulbar palsy, limb apraxia, dystonia, cortical sensory loss, and tremor.

Conditions to be treated by the present compositions and methods also include, but are not limited to, spinal muscular atrophy (SMA), amytrophic lateral sclerosis (ALS), spinal bulbar muscular atrophy (SBMA), spinal cerebellar ataxia, primary lateral sclerosis (PLS), or traumatic spinal cord injury, primary lateral sclerosis (PLS), progressive muscular atrophy (PMA), hereditary spastic paraparesis (HSP), X-linked spinobulbar muscular atrophy (SBMA; Kenney disease), progressive bulbar palsy, pseudo-bulbar palsy, post-polio syndrome (PPS), Huntington's disease, Essential tremor (ET), paralysis, and Parkinson's disease.

### Recombinant AAV Vectors

In certain embodiments, the nucleic acid is provided in a recombinant adeno-associated virus (AAV) vector. In additional embodiments, the AAV vector further comprises a chicken Beta-actin promoter and wherein the AAV is capable of crossing the blood-brain barrier (BBB). In yet additional embodiments, the AAV is AAV-PHP.eB, AAV8 or AAV9.

For example, an AAV vector may be administered at or near the axon terminals of neurons. The neurons internalize the AAV vector and transport it in a retrograde manner along the axon to the cell body. Similar properties of adenovirus, HSV, and pseudo-rabies virus have been shown to deliver genes to distal structures within the brain (Soudas et al. (2001) FASEB J. 15:2283-2285; Breakefield et al. (1991) New Biol. 3:203-218; and deFalco et al. (2001) Science, 291:2608-2613).

In aspects where gene transfer is mediated by a DNA viral vector, such as an adenovirus (Ad) or adeno-associated virus (AAV), a vector construct refers to the polynucleotide comprising the viral genome or part thereof, and a transgene. Suitable neurotrophic viral vectors for the practice of this invention include, but are not limited to adeno-associated viral vectors (AAV), herpes simplex viral vectors and lentiviral vectors.

AAV of any serotype can be used. The serotype of the viral vector used in certain embodiments of the invention may be AAV1, AAV2, AAV3, AAV4, AAV5, AAV6, AAV7, AAV8, and AAV9 (see, e.g., Gao et al. (2002) PNAS, 99:11854-11859; and Viral Vectors for Gene Therapy: Methods and Protocols, ed. Machida, Humana Press, 2003). Other serotype besides those listed herein can be used. Furthermore, pseudotyped AAV vectors may also be utilized in the methods described herein. Pseudotyped AAV vectors are those which contain the genome of one AAV serotype in the capsid of a second AAV serotype; for example, an AAV vector that contains the AAV2 capsid and the AAV1 genome or an AAV vector that contains the AAV5 capsid and the AAV 2 genome (Auricchio et al., (2001) Hum. Mol. Genet., 10(26):3075-81).

In certain embodiments, the concentration or titer of the vector in the composition is at least: (a) 5, 6, 7, 8, 9, 10, 15, 20, 25, or 50 (×10¹² gp/ml); (b) 5, 6, 7, 8, 9, 10, 15, 20, 25, or 50 (×10⁹ to/ml); or (c) 5, 6, 7, 8, 9, 10, 15, 20, 25, or 50 (×10¹⁰ iu/ml).

In experimental mice, the total volume of injected AAV solution is for example, between 1 to 20 μl. For other mammals, including the human brain, volumes and delivery rates are appropriately scaled. For example, it has been demonstrated that volumes of 150 μl can be safely injected in the primate brain (Janson et al. (2002) Hunt. Gene Ther. 13:1391-1412). Treatment may consist of a single injection per target site, or may be repeated in one or more ventricles. Suitable ventricles include the lateral ventricles, third ventricle, and the fourth ventricle. Multiple injection sites can be used. For example, in some embodiments, in addition to the first administration site, a composition containing a viral vector carrying a transgene is administered to another site which can be contralateral or ipsilateral to the first administration site. Injections can be single or multiple, unilateral or bilateral.

In addition to the elements identified above for recombinant AAV vectors, the vector may also include conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

As used herein, a nucleic acid sequence and regulatory sequences are said to be operably linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., shRNA, miRNA).

For nucleic acids encoding proteins, a polyadenylation sequence generally is inserted following the transgene sequences and before the 3′ AAV ITR sequence. An AAV construct useful in the present invention may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available (sec, e.g., Sambrook et al, and references cited therein at, for example, pages 3.18 3.26 and 16.17 16.27 and Ausubel et al., Current Protocols in Molecular Biology, John Wiley & Sons, New York, 1989). In some circumstances, a Foot and Mouth Disease Virus 2A sequence may be included in a polyprotein; this is a small peptide (approximately 18 amino acids in length) that has been shown to mediate the cleavage of polyproteins. The cleavage activity of the 2A sequence has previously been demonstrated in artificial systems including plasmids and gene therapy vectors (AAV and retroviruses) (Ryan et al., EMBO, 1994; 4:928-933; Mattion et al., J Virology, November 1996; p. 8124-8127; Furler et al., Gene Therapy, 2001; 8:864-873; and Halpin et al., The Plant Journal, 1999; 4:453-459; de Felipe et al., Gene Therapy, 1999; 6:198-208; de Felipe et al., Human Gene Therapy, 2000; 11:1921-1931.; and Klump et al., Gene Therapy, 2001; 8:811-817).

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors may optionally include 5′ leader or signal sequences.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) (sec, e.g., Boshart et al, Cell, 41:521-530 (1985)), the SV40 promoter, the dihydrofolate reductase promoter, the 13-actin promoter, the phosphoglycerol kinase (PGK) promoter, and the EF1a promoter.

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al. (1992) Proc. Natl. Acad. Sci. USA, 89:5547-5551), the tetracycline-inducible system (Gossen et al. (1995) Science, 268:1766-1769, see also Harvey et al. (1998) Curr. Opin. Chem. Biol., 2:512-518), the RU486-inducible system (Wang et al. (1997) Nat. Biotech., 15:239-243 and Wang et al. (1997) Gene Ther., 4:432-441) and the rapamycin-inducible system (Magari et al. (1997) J. Clin. Invest., 100:2865-2872). Still other types of inducible promoters which may be useful in this context are those which are regulated by a specific physiological state, e.g., temperature, acute phase, a particular differentiation state of the cell, or in replicating cells only.

In another embodiment, the native promoter, or fragment thereof, for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers) are well known in the art. Exemplary tissue-specific regulatory sequences include but are not limited to the following tissue specific promoters: neuronal such as neuron-specific enolase (NSE) promoter (Andersen et al. (1993) Cell. Mol. Neurobiol., 13:503-15), neurofilament light-chain gene promoter (Piccioli et al. (1991) Proc. Natl. Acad. Sci. USA, 88:5611-5), and the neuron-specific vgf gene promoter (Piccioli et al. (1995) Neuron, 15:373-84). In some embodiments, the tissue-specific promoter is a promoter of a gene selected from: neuronal nuclei (NeuN), glial fibrillary acidic protein (GFAP), adenomatous polyposis coli (APC), and ionized calcium-binding adapter molecule 1 (Iba-1). In some embodiments, the promoter is a chicken Beta-actin promoter.

Methods for obtaining recombinant AAVs having a desired capsid protein have been described (See, for example, US 2003/0138772, the contents of which are incorporated herein by reference in their entirety). A number of different AAV capsid proteins have been described, for example, those disclosed in Gao et al. (2004) J. Virol, 78(12):6381-6388; Gao et al. (2004) Proc Natl Acad Sci USA, 100(10):6081-6086. For the desired packaging of the presently described constructs and methods, the AAV9 vector and capsid is preferred. However, it is noted that other suitable AAVs such as rAA Vrh.8 and rAA Vrh. 10, or other similar vectors may be adapted for use in the present invention. Typically the methods involve culturing a host cell which contains a nucleic acid sequence encoding an AAV capsid protein or fragment thereof; a functional rep gene; a recombinant AAV vector composed of AAV inverted terminal repeats (ITRs) and a transgene; and sufficient helper functions to permit packaging of the recombinant AAV vector into the AAV capsid proteins.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters.

The recombinant AAV vector, rep sequences, cap sequences, and helper functions for producing the AAV may be delivered to the packaging host cell using any appropriate genetic element (vector). The selected genetic clement may be delivered by any suitable method, including those described herein. See, e.g., Fisher et al. (1993) J. Virol., 70:520-532 and U.S. Pat. No. 5,478,745.

In some embodiments, recombinant AAVs may be produced using the triple transfection method (e.g., as described in detail in U.S. Pat. No. 6,001,650). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). Non-limiting examples of vectors suitable for use with the present invention include pHLP19, described in U.S. Pat. No. 6,001,650 and pRep6cap6 vector, described in U.S. Pat. No. 6,156,303. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

### Pharmaceutical Compositions and Administration

The pharmaceutical compositions can further comprise one or more pharmaceutically acceptable excipient, ligand, a conjugate, a vector, a lipid, a nanoparticle, a liposome, a carrier, an adjuvant or a diluent.

Delivery vehicles such as liposomes, nanocapsules, microparticles, microspheres, lipid particles, vesicles, and the like can be used to deliver the nucleic acid molecules described herein.

The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868; and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 ∥m. Sonication of MLVs results in the formation of small unilamellar vesicles (SUVs) with diameters in the range of 200 to 500 Å, containing an aqueous solution in the core.

Alternatively, nanocapsule or nanoparticle formulations may be used. Nanocapsules can generally entrap substances in a stable and reproducible way. Nanoparticles can be used to transport drugs through the BBB when administered intravenously as well as the factors that influence its transportation.

NPs are colloidal carriers that can have a natural or synthetic origin and can vary from 1 to 1000 nm in size. Synthetic NPs may be prepared from polymeric materials such as poly(ethylenimine) (PEI), poly(alkylcyanoacrylates), poly(amidoamine) dendrimers (PAMAM), poly(ε-caprolactone) (PCL), poly(lactic-co-glycolic acid) (PLGA), polyesters (poly (lactic acid) (PLA), or from inorganic materials such as gold, silicon dioxide (silica), among others. These carriers can transport drugs by adsorbing, entrapping or bounding covalently to them. Natural NPs are produced from natural polymers, such as polysaccharides (chitosan and alginate), amino acids (poly (lysine), poly (aspartic acid) (PASA)), or proteins (gelatin and albumin). Natural NPs have the advantage of providing biological signals to interact with specific receptors/transporters expressed by endothelial cells.

A number of ligands have been conjugated to NPs to facilitate BBB penetration. Such molecules can be grouped into four different types: (i) ligands that mediate the adsorption of proteins from the bloodstream that interact directly with BBB receptors or transporters; (ii) ligands that have direct interaction per se with BBB receptors or transporters; (iii) ligands that increase charge and hydrophobicity; and (iv) ligands that improve blood circulation time (e.g. PEG).

Other methods for assisting the NPs to cross the blood-brain barrier would include but are not limited to receptor mediated transport, transporter mediated transport, absorptive mediated transport, and cell penetrating transport.

Mammalian virus vectors that can be used to deliver RNA include oncoretroviral vectors, adenovirus vectors, Herpes simplex virus vectors, and lentiviruses.

In particular, HSV vectors are tropic for the central nervous system (CNS) and can establish lifelong latent infections in neurons.

The AAVs may be delivered to a subject in compositions according to any appropriate methods known in the art. The AAV, preferably suspended in a physiologically compatible carrier (e.g., in a composition), may be administered to a subject, e.g., a human, mouse, rat, cat, dog, sheep, rabbit, horse, cow, goat, pig, guinea pig, hamster, chicken, turkey, or a non-human primate. In certain embodiments, compositions may comprise an AAV alone, or in combination with one or more other viruses (e.g., a second AAV encoding having one or more different transgenes).

Suitable carriers may be readily selected by one of skill in the art in view of the indication for which the AAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present invention.

Optionally, the compositions of the invention may contain, in addition to the AAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, parabens, ethyl vanillin, glycerin, phenol, and parachlorophenol. Suitable chemical stabilizers include gelatin and albumin.

The dose of AAV virions required to achieve a desired effect or “therapeutic effect,” e.g., the units of dose in vector genomes/per kilogram of body weight (vg/kg), will vary based on several factors including, but not limited to: the route of AAV administration, the level of gene or RNA expression required to achieve a therapeutic effect, the specific disease or disorder being treated, and the stability of the gene or RNA product. One of skill in the art can readily determine an AAV virion dose range to treat a subject having a particular disease or disorder based on the aforementioned factors, as well as other factors that are well known in the art. An effective amount of the AAV is generally in the range of from about 10 μl to about 100 ml of solution containing from about 10⁹ to 10¹⁶ genome copies per subject. Other volumes of solution may be used. The volume used will typically depend, among other things, on the size of the subject, the dose of the AAV, and the route of administration. For example, for intrathecal or intracerebral administration a volume in range of 1 μl to 10 μl or 10 μl to 100 μl may be used. For intravenous administration a volume in range of 10 μl to 100 μl, 100 μl to 1 ml, 1 ml to 10 ml, or more may be used. In some cases, a dosage between about 10¹⁰ to 10¹² AAV genome copies per subject is appropriate. In certain embodiments, 10¹² AAV genome copies per subject is effective to target CNS tissues. In some embodiments the AAV is administered at a dose of 10¹⁰, 10¹¹, 10¹², 10¹³, 10¹⁴, or 10¹⁵ genome copies per subject. In some embodiments the AAV is administered at a dose of 10¹⁰, 10¹¹, 10¹², 10¹³, or 10¹⁴ genome copies per kg.

In some embodiments, AAV compositions are formulated to reduce aggregation of AAV particles in the composition, particularly where high AAV concentrations are present (e.g., about 10¹³ GC/ml or more). Methods for reducing aggregation of AAVs are well known in the art and, include, for example, addition of surfactants, pH adjustment, salt concentration adjustment, etc. (See, e.g., Wright et al. (2005) Molecular Therapy 12:171-178.)

Formulation of pharmaceutically acceptable excipients and carrier solutions is well-known to those of skill in the art, as is the development of suitable dosing and treatment regimens for using the particular compositions described herein in a variety of treatment regimens. Typically, these formulations may contain at least about 0.1% of the active ingredient or more, although the percentage of the active ingredient(s) may, of course, be varied and may conveniently be between about 1 or 2% and about 70% or 80% or more of the weight or volume of the total formulation. Naturally, the amount of active ingredient in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition). Some variation in dosage will necessarily occur depending on the condition of the host. The person responsible for administration will, in any event, determine the appropriate dose for the individual host.

Sterile injectable solutions are prepared by incorporating the active AAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The AAV compositions disclosed herein may also be formulated in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the AAV compositions of the present invention into suitable host cells. In particular, the AAV vector delivered components may be formulated for delivery either encapsulated in a lipid particle, a liposome, a vesicle, a nanosphere, or a nanoparticle or the like.

In addition to the methods of delivery described above, the following techniques are also contemplated as alternative methods of delivering the AAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Patent. No. 5,779,708), microchip devices (U.S. Pat. No. 5,797,898), ophthalmic formulations, transdermal matrices (U.S. Pat. Nos. 5,770,219 and 5,783,208) and feedback-controlled delivery (U.S. Pat. No. 5,697,899).

To prepare the present pharmaceutical compositions, a conjugate, a vector, a lipid, a nanoparticle, a liposome, an adjuvant or a diluent may be further admixed with a pharmaceutically acceptable carrier or excipient. Sec, e.g., Remington's Pharmaceutical Sciences and U.S. Pharmacopeia: National Formulary, Mack Publishing Company, Easton, PA (1984).

Formulations of therapeutic agents may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY).

Toxicity and therapeutic efficacy of the therapeutic compositions, administered alone or in combination with another agent, can be determined by standard pharmaceutical procedures in cell cultures or experimental animals, e.g., for determining the LD₅₀ (the dose lethal to 50% of the population) and the ED50 (the dose therapeutically effective in 50% of the population). The dose ratio between toxic and therapeutic effects is the therapeutic index (LD₅₀/ED₅₀). In particular aspects, therapeutic compositions exhibiting high therapeutic indices are desirable. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration.

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial.

In particular embodiments, the composition or therapeutic can be administered by an invasive route such as by injection. In further embodiments of the invention, the composition, therapeutic, or pharmaceutical composition thereof, is administered intravenously, subcutaneously, intramuscularly, intraarterially, intra-articularly (e.g. in arthritis joints), intratumorally, or by inhalation, acrosol delivery. Administration by non-invasive routes (e.g., orally; for example, in a pill, capsule or tablet) is also within the scope of the present invention.

In order to overcome any issue of the pharmacological agents crossing the blood/brain barrier, intrathecal administration is a further preferred form of administration. Intrathecal administration involves injection of the drug into the spinal canal, more specifically the subarachnoid space such that it reaches the cerebrospinal fluid. This method is commonly used for spinal anesthesia, chemotherapy, and pain medication. Intrathecal administration can be performed by lumbar puncture (bolus injection) or by a port-catheter system (bolus or infusion). The catheter is most commonly inserted between the laminae of the lumbar vertebrae and the tip is threaded up the thecal space to the desired level (generally L3-L4). Intrathecal formulations most commonly use water, and saline as excipients but EDTA and lipids have been used as well.

Compositions can be administered with medical devices known in the art. For example, a present pharmaceutical composition can be administered by injection with a hypodermic needle, including, e.g., a prefilled syringe or autoinjector.

The present pharmaceutical compositions may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556.

Alternately, one may administer the pharmaceutical composition in a local rather than systemic manner, for example, via injecting directly into the desired target site, often in a depot or sustained release formulation. Furthermore, one may administer the composition in a targeted drug delivery system, for example, in a liposome coated with a tissue-specific antibody, targeting, for example, the brain. The liposomes will be targeted to and taken up selectively by the desired tissue.

The administration regimen depends on several factors, including the serum or tissue turnover rate of the therapeutic composition, the level of symptoms, and the accessibility of the target cells in the biological matrix. Preferably, the administration regimen delivers sufficient therapeutic composition to effect improvement in the target disease state, while simultaneously minimizing undesired side effects. Accordingly, the amount of biologic delivered depends in part on the particular therapeutic composition and the severity of the condition being treated.

Determination of the appropriate dose is made by the clinician, e.g., using parameters or factors known or suspected in the art to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. In general, it is desirable that a biologic that will be used is derived from the same species as the animal targeted for treatment, thereby minimizing any immune response to the reagent.

As used herein, the terms “therapeutically effective amount”, “therapeutically effective dose” and “effective amount” refer to an amount of the present nucleic acid molecules, mutant proteins/polypeptides, and/or modulators that, when administered alone or in combination with an additional therapeutic agent to a cell, tissue, or subject, is effective to cause a measurable improvement in one or more symptoms of a disease or condition or the progression of such disease or condition. A therapeutically effective dose further refers to that amount of the agent sufficient to result in at least partial amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity. The present agents/compositions may prevent or delay onset or amelioration of symptoms of the condition in a subject or an attainment of a desired biological outcome, such as correction of neuropathology, e.g., cellular pathology associated with a neurodegenerative disease.

### Kits

The present invention also provides kits comprising the present composition/agent (nucleic acid molecules, variant or mutant proteins/polypeptides, and/or modulators) in kit form. A kit of the present invention includes one or more components described herein, in association with one or more additional components including, but not limited to a pharmaceutically acceptable ligand, a conjugate, a vector, a lipid, a nanoparticle, a liposome, an adjuvant, a diluent, carrier or excipient.

If the kit includes a pharmaceutical composition for parenteral administration to a subject, the kit can include a device for performing such administration. For example, the kit can include one or more hypodermic needles or other injection devices as discussed above.

The kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10% or 20%.

The present invention may be better understood by reference to the following non-limiting examples, which are presented in order to more fully illustrate the preferred embodiments of the invention. They should in no way be construed to limit the broad scope of the invention.

### Example 1

Reduced survival motor neuron (SMN) protein triggers the motor neuron disease, spinal muscular atrophy (SMA). Restoring SMN prevents disease, but it is not known how neuromuscular function is preserved. We used model mice to map and identify an Hspa8^{G470R} synaptic chaperone variant, which suppressed SMA. Expression of the variant in the severely affected mutant mice increased lifespan >10-fold, improved motor performance, and mitigated neuromuscular pathology. Mechanistically, Hspa8^{G470R} altered SMN2 splicing and simultaneously stimulated formation of a tripartite chaperone complex, critical for synaptic homeostasis, by augmenting its interaction with other complex members. Concomitantly, synaptic vesicular SNARE complex formation, which relies on chaperone activity for sustained neuromuscular synaptic transmission, was found perturbed in SMA mice and patient-derived motor neurons and was restored in modified mutants. Identification of the Hspa8^{G470R} SMA modifier implicates SMN in SNARE complex assembly and casts new light on how deficiency of the ubiquitous protein causes motor neuron disease.

We previously generated a severely affected, humanized mouse model of SMA.¹⁶ Consistent with observations of discordant SMA sibs and the notion of disease modifiers, we found that distinct mouse strain backgrounds altered phenotypic severity in the mutants. Here, we report the mapping, identification, and functional significance of a novel SMA modifier associated with the altered phenotypes. The modifier, a G470R variant of the synaptic chaperone, Hspa8, dramatically suppresses severe disease. Investigations of the mechanism of action of Hspa8^{G470R} led to the discovery that SNARE complex assembly, a process reliant on Hspa8 chaperone activity and crucial for neurotransmission, is impaired at the neuromuscular synapses of SMA mice but restored in mutants expressing the modifier.

Our results suggest that SMN functions to facilitate assembly of synaptic complexes critical to neuromuscular function.

### RESULTS

### Evidence of a Genetic Modifier of the Mouse SMA Phenotype

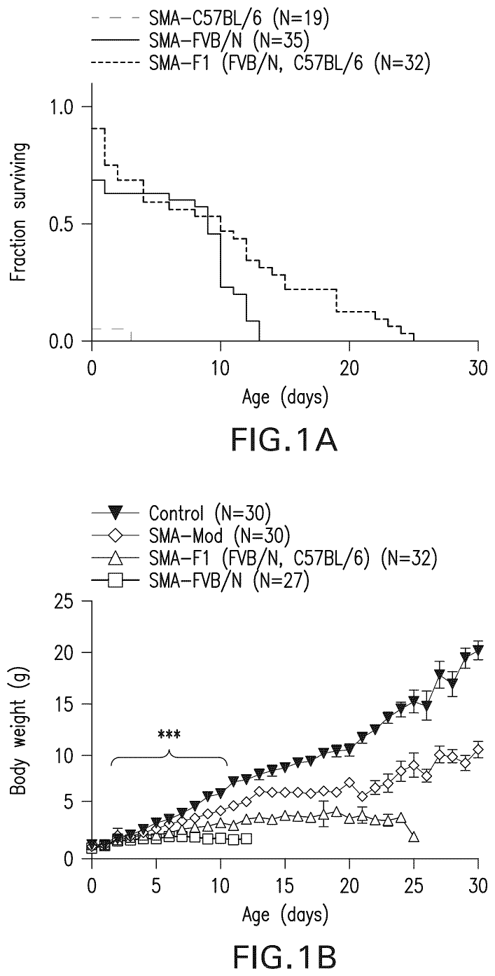

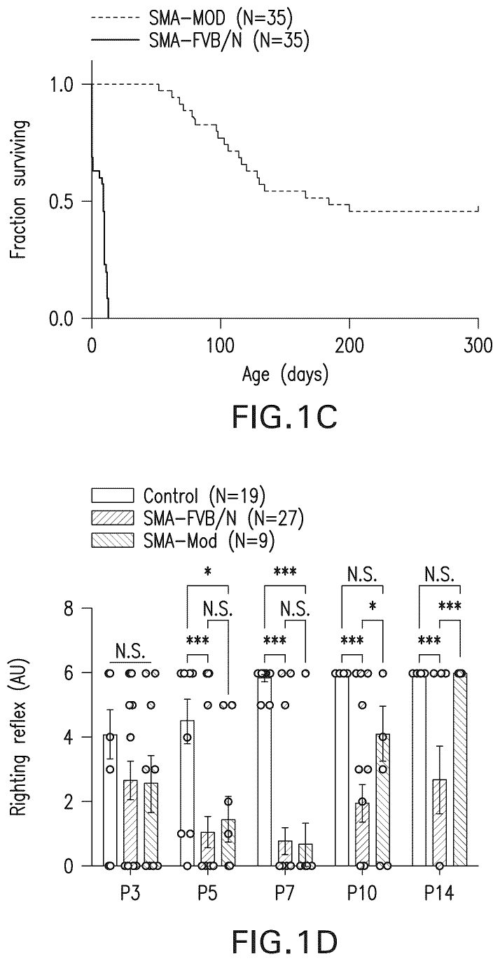

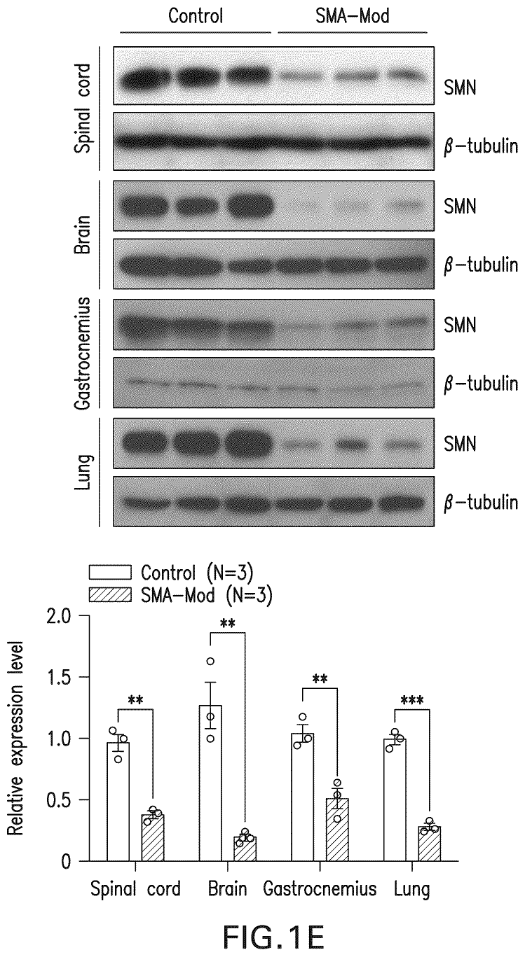

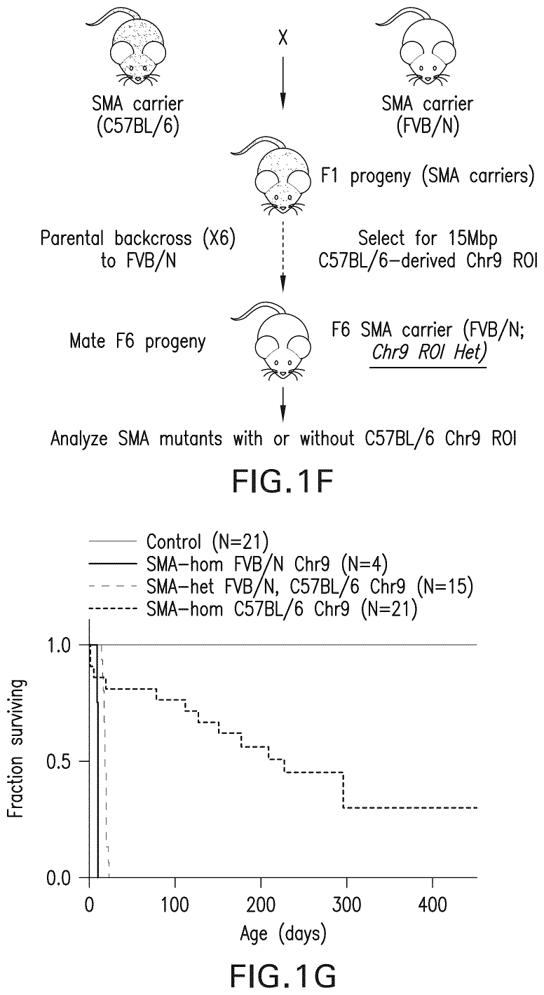

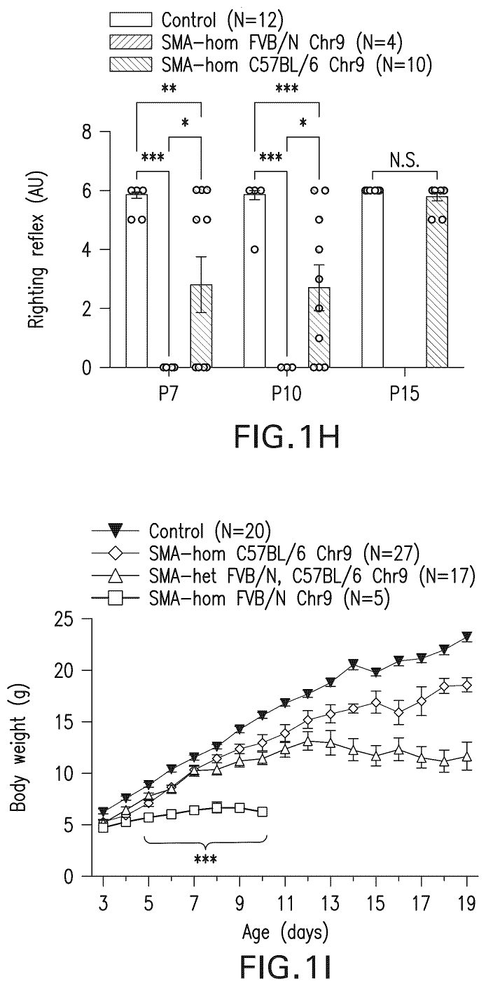

We previously developed severe (“Δ7”) SMA model mice.¹⁶ On a mixed (FVB/N×C57BL/6) background, median and maximum lifespans of the mutants (SMN2^{+/+}; SMND7^{+/+}; Smn^{-/-}) were about 11 and 25 days, respectively. These were reduced on the pure parental strains (FIG. 1A). Consistent with their shortened lifespans, FVB/N SMA mutants were significantly smaller than were those on the mixed background (FIG. 1B), suggestive of the existence of protective modifiers in one or both parental strains. To map the modifiers, a classic F2 intercross was employed to produce SMA mutants. Most mutants, thus, derived exhibited the typically severe SMA phenotype, succumbing to disease at approximately PND14. However, about 6% presented with an unexpectedly mild phenotype characterized by a maximum lifespan exceeding 300 days (FIG. 1C). This dramatic extension of the 2-week lifespan of typically severe SMA mice was accompanied by an increase in body weight (FIG. 1B) and a complete recovery, as assessed in righting ability at PND14, of the paralytic SMA phenotype (FIG. 1D). Overtly, the mice remained active into adulthood but developed necrotic tails. Still, the results strongly suggested the disease-modifying effects of an SMA suppressor.

We examined the modified mutants for SMN levels and evidence of neuromuscular pathology classically seen in SMA.¹⁷⁻¹⁹ At PND70, the mutants continued to express acutely low SMN protein (FIG. 1E). Yet, morphometric counts of spinal motor neurons revealed no difference between the modified mutants and healthy controls and, consistent with this finding, there was neither a change in mutant spinal motor neuron soma size nor evidence of deafferentiation. An examination of two muscles, the gastrocnemius and triceps, did not uncover any group atrophy of the fibers but, congruent with the smaller size of the mutants, did reveal a uniform drop in fiber size. Δ7 SMA mice have defective neuromuscular junctions (NMJs) characterized by small, immature endplates bereft of the perforations observed in wild-type (WT) NMJs.²⁰⁻²² These defects were partially rescued in the modified mutants; the endplates remained smaller than in controls but appeared perforated and fully mature. Moreover, intracellular recordings to gauge NMJ function indicated that neurotransmission in the EDL muscle was normalized in the modified mutants. These observations suggested that the modifier responsible for extending lifespan in our mutants also mitigated the neuromuscular defects typical of SMA.

Δ7 mutants are known to suffer cardiac abnormalities.²³ We investigated if such dysfunction was also resolved in our F2 SMA modified mutants. Echocardiography indicated that cardiac function was indeed normalized in the mutants. Consistent with this observation, gross cardiac structure and heart to body mass ratios were normal. Collectively, these preliminary findings suggested that disease modification was systemic, involving cardiac function too.

### The SMA Modifier is Mapped to Mouse Chromosome 9

To reveal the chromosomal location of the SMA modifier, a cohort of F2 mutants exhibiting either the severe or the mild phenotype was subjected to a genome-wide scan using approximately 180 and approximately 730 single nucleotide polymorphic (SNP) markers. We found linkage between the modified phenotype and a region of chromosome 9 (Chr.9) deriving from C57BL/6 and extending from 34 to 49 Mbp. To ascertain if this region of Chr.9 from the C57BL/6 strain was sufficient to produce the mild phenotype, we introduced it via successive backcrosses and SNP-based selection into SMA carriers deriving the rest of their genomes from the FVB/N strain. Such carriers, heterozygous for the region of interest (ROI), were then bred to produce mutants (FIG. 1F). As expected, mutants homozygous FVB/N for the ROI were very severely affected and perished on or before PND10. In contrast, mutants heterozygous at the ROI exhibited a median survival of 18 days, and mice deriving both copies of the Chr.9 ROI from C57BL/6 lived much longer (FIG. 1G). This suggested that the region of Chr. 9 being investigated did indeed harbor an SMA suppressor and, furthermore, that its disease-mitigating effects were dose dependent. Consistent with the longer lifespans of mutants harboring the ROI, we found that motor performance in the mice was improved and body weight enhanced relative to severely affected SMA mice (FIGS. 1H and 1I). Investigations of muscle fiber morphology and spinal motor neuron counts confirmed the modifying effects of the Chr.9 ROI. At PND7, myofiber area frequency distribution analysis showed that fibers of mutants homozygous for the modifying ROI were closer in size to that of controls, and this was borne out when average fiber size was quantified. Fibers from mutants with the modifying

ROI were significantly larger than those from mutants devoid of it, although they remained smaller than fibers in controls. By 6 months, differences in fiber size between modified mutants and controls were no longer detectable and, morphologically, the mutant muscles appeared healthy. Motor neuron counts at this stage failed to detect significant difference between controls and mutants homozygous for the ROI.

### A G470R Variant in the Synaptic Chaperone Hspa8 is the SMA Modifier

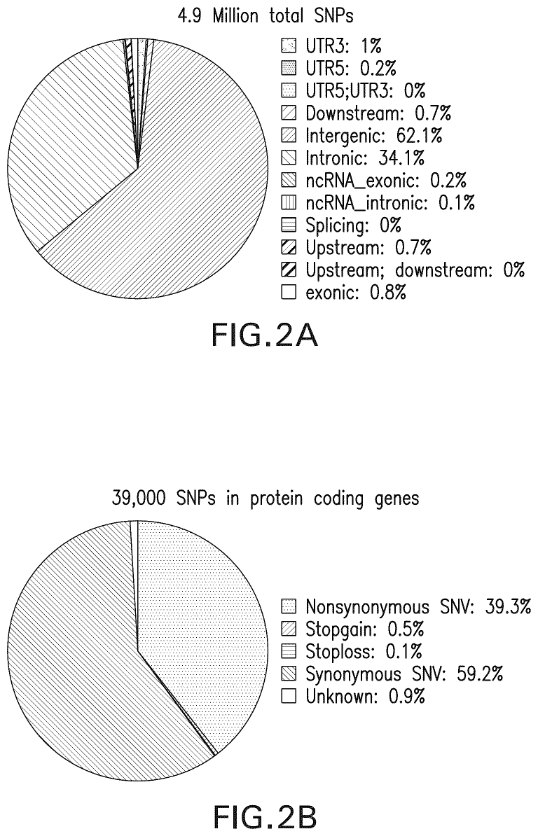

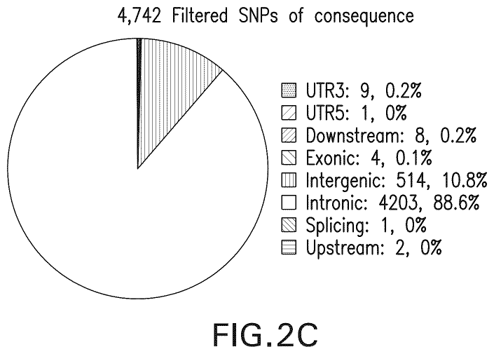

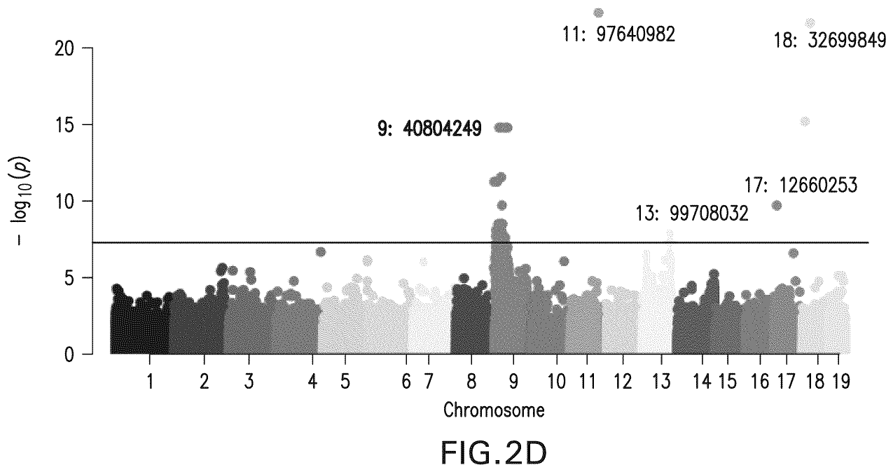

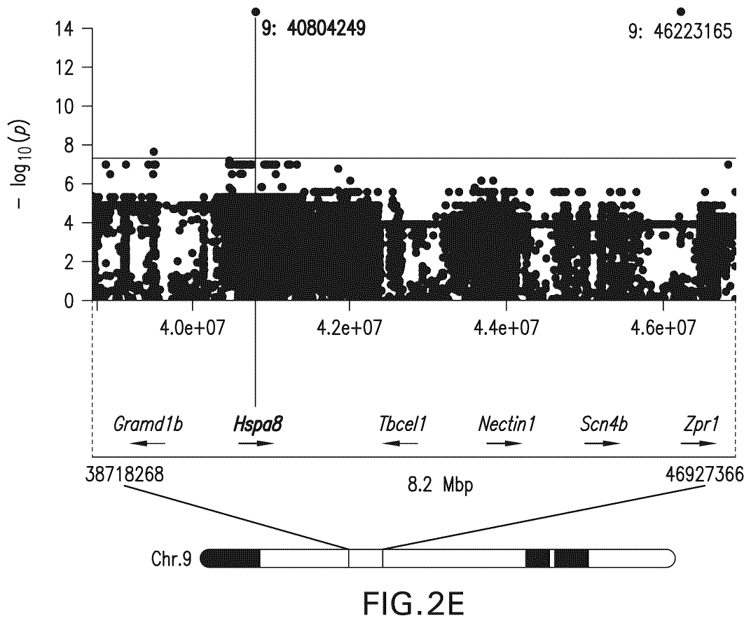

To ascertain the precise identity of the SMA modifier, a second cohort of F2 mutants, similar to those employed to localize it chromosomally, was subjected to whole genome sequencing and variants specifically in the Chr.9 ROI examined in detail. Our rough localization studies predicted that fully modified mutants, defined as those with lifespans of >50 days, would be homozygous for the C57BL/6 version of the modifier, whereas typically severe mutants, succumbing to disease before PND14, would inherit both modifier alleles from the FVB/N strain. Mutants heterozygous for the modifier were expected to constitute a third group with an intermediate phenotype. We furthermore focused on non-synonymous changes in protein-coding genes. A genome-wide association study (GWAS) revealed approximately 4.9 million total SNP variants, 0.8% of which were in protein-coding regions (FIGS. 2A and 2B). After filtering for stringency, 4,742 significant SNPs emerged from the GWAS. The preponderance of these mapped to the Chr.9 ROI (FIGS. 2C-2E) and were non-exonic; only 2 were non-synonymous variants. One of these resulted in a T1309M alteration in IGSF9b, a protein expressed in synapses.²⁴ However, the residue is not evolutionarily conserved and was rejected. The second variant of interest, a G/C nucleotide change, caused a G470R substitution in Hspa8 and piqued our curiosity for several reasons. First, the transversion is not evident in the ancestral C57BL/6 strain, suggesting that it arose de novo in our C57BL/6 colony. Second, Hspa8 is a chaperone, important for synaptic proteostasis and neurotransmission,^{25.26} that functions disrupted in SMA. Finally, the glycine at position 470 is a constituent of a 10 amino acid IPPAPRGVPQ (SEQ ID NO:1) motif, perfectly conserved from worms to humans suggesting an important function.

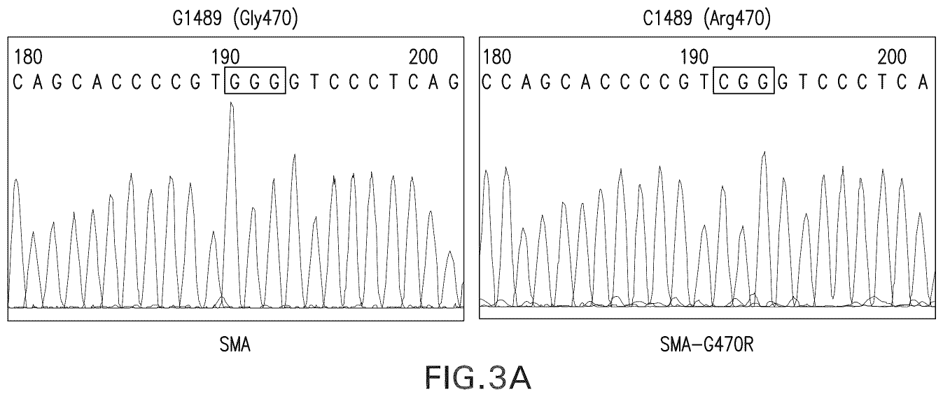

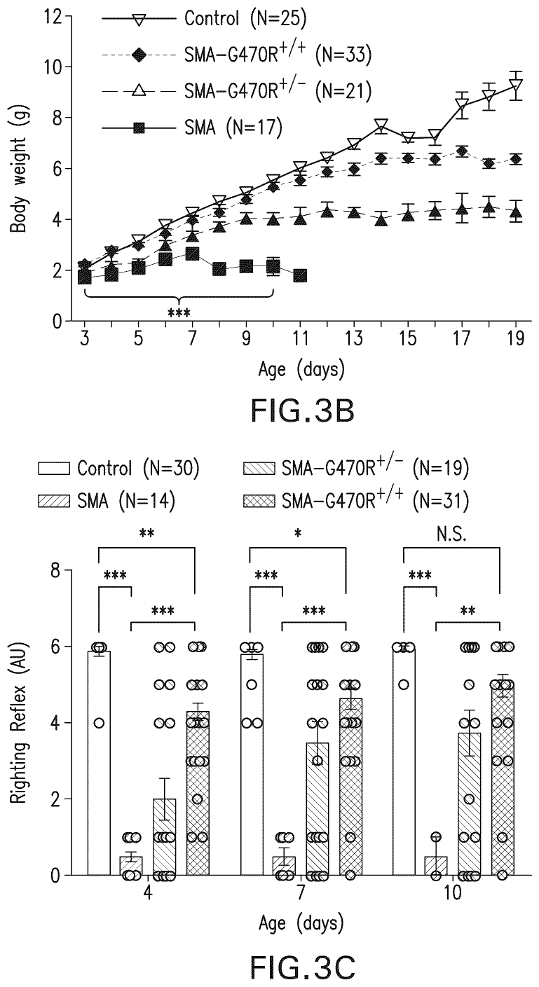

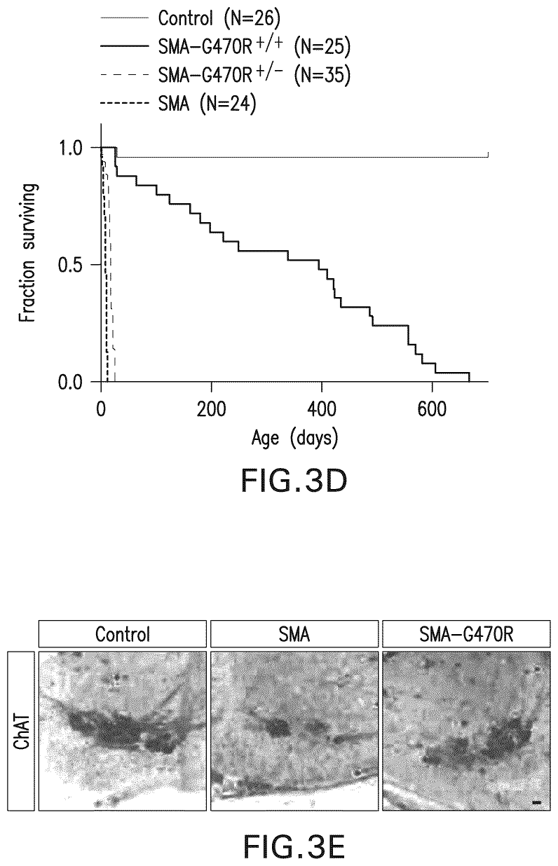

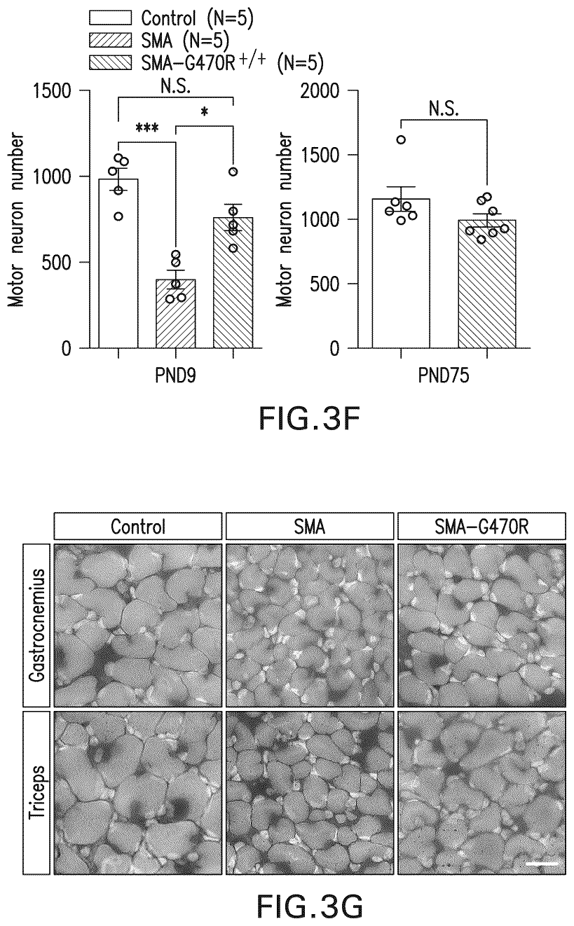

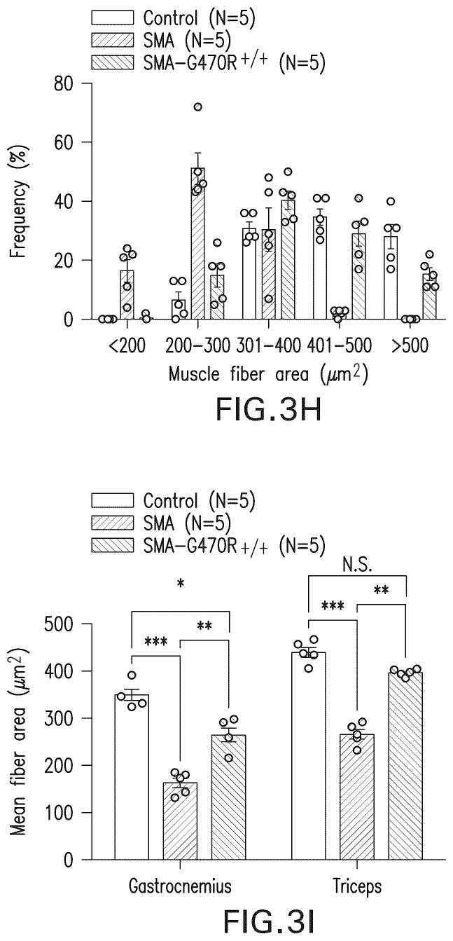

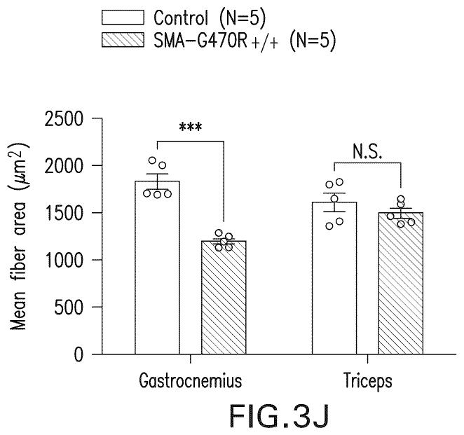

To demonstrate unequivocally that Hspa8^{G470R} underlies the modified SMA phenotype, we edited the wild-type GGG (Gly470) codon to CGG—for Arginine—in FVB/N-derived Δ7 carriers (FIG. 3A). Two independent lines termed C3 and C5 were, thus, obtained. Each was used to generate SMA mutants. Consistent with Hspa8^{G470R} being the SMA-modifying factor, we found that median survival of mutants from the two lines was increased vis-à-vis typically affected SMA mice, although lifespans of the modified mutants did not differ substantially from each other. The latter observation suggested that the two lines were equivalent, and this was reflected in similar body weights, motor performance, and relative levels of SMN and Hspa8 in the mutants. Importantly, we also showed equivalent amounts of Hspa8 in animals with or without G470R, suggesting that the variant does not alter protein stability. Considering the equivalence between the C3 and C5 lines, all subsequent analyses were conducted on mutants from the C5 line.

### Hspa8^{G470R} Mitigates Neuromuscular Pathology in SMA Model Mice

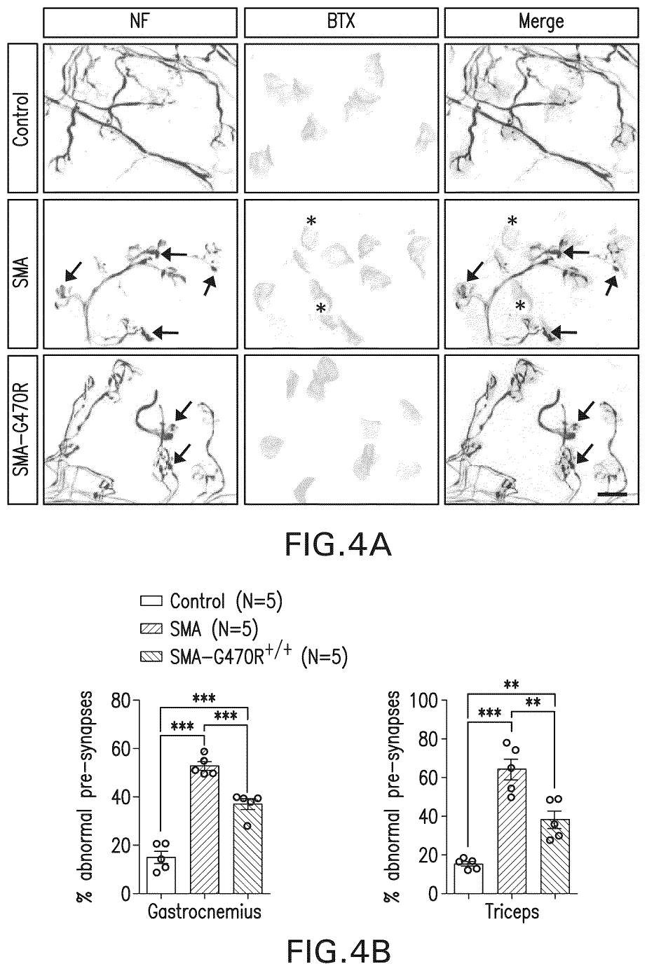

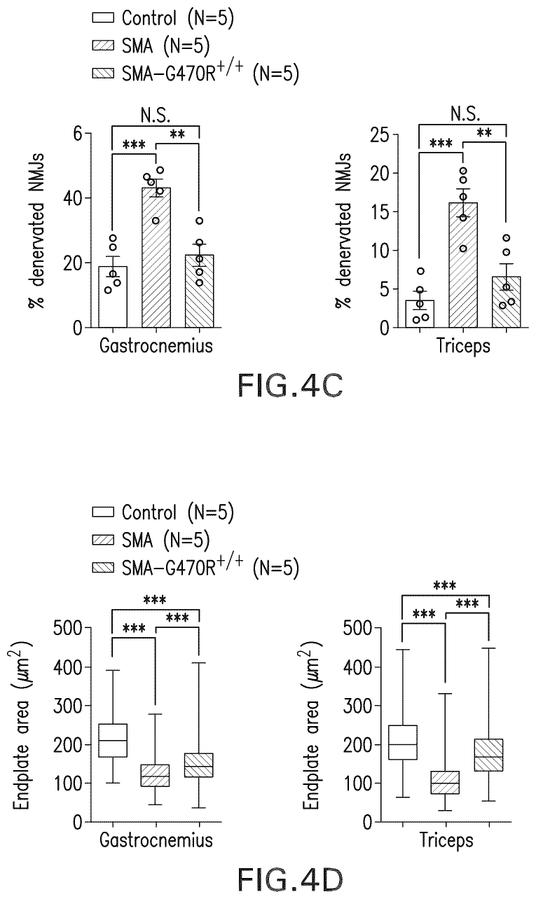

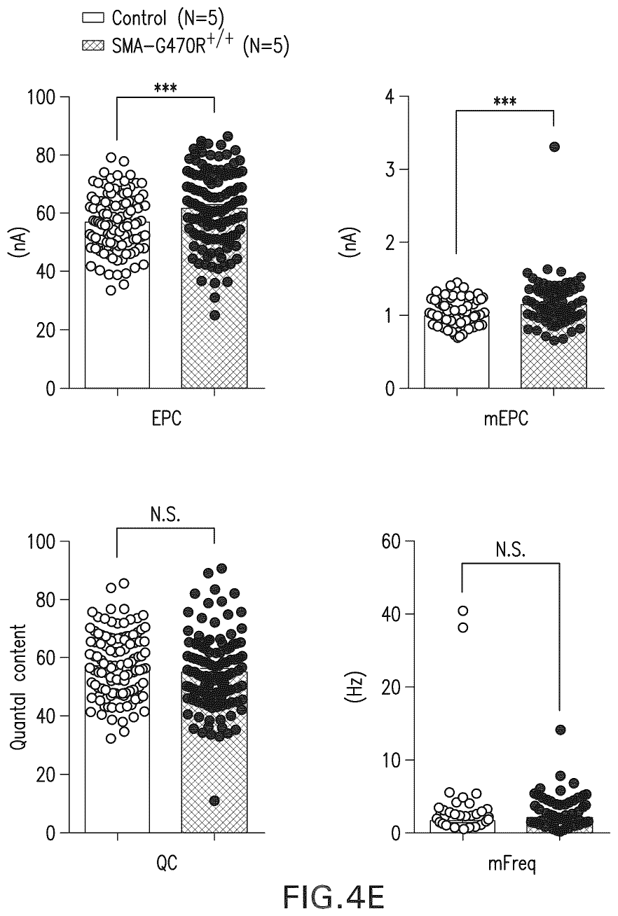

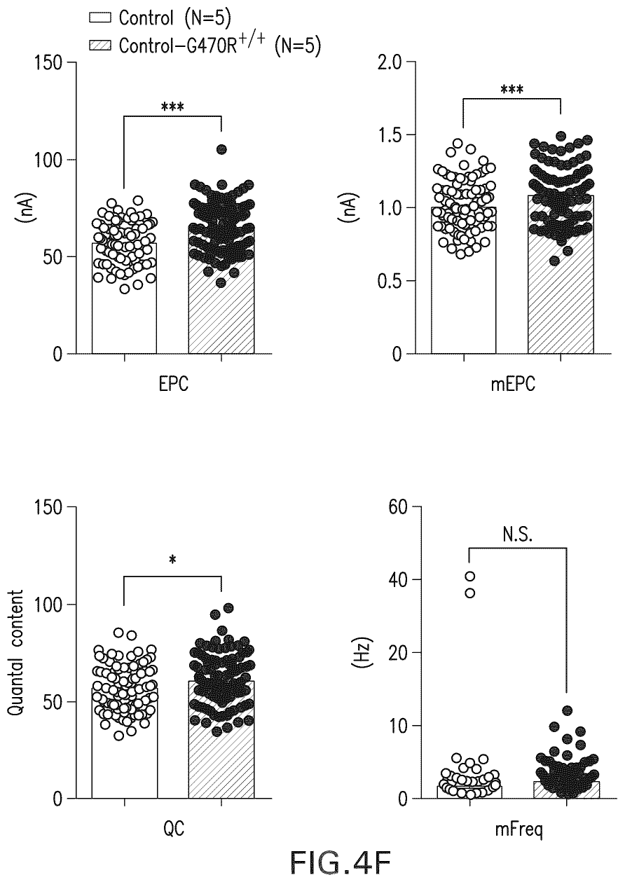

We next investigated how Hspa8^{G470R} might mitigate neuromuscular dysfunction. Expectedly, we found that mutants harboring the variant were larger, more agile, and considerably longer-lived than mutants expressing WT Hspa8 (FIGS. 3B-3D). The disease-modifying effects of the G470R variant were furthermore confirmed in a more severe model of SMA, suggesting that the modification is not line-specific. Moreover, on the Δ7 background, Hspa8^{G470R} expression restored spinal motor neuron numbers in PND9 and PND75 mutants and resulted in muscle fibers that were larger than those of age-matched severe SMA mice expressing WT Hspa8 (FIGS. 3E-3J). An examination of the NMJs, which in severe SMA mice feature small, immature, denervated endplates juxtaposed against nerve terminals engorged with neurofilament (NF) protein, revealed significantly fewer of these defects in the presence of Hspa8^{G470R} (FIGS. 4A-4D). This was accompanied by restoration of function, as assessed electro-physiologically, in hindlimb muscles of adult, modified mutants (FIG. 4E). In fact, consistent with a role for Hspa8 in neurotransmission, end-plate currents in Hspa8^{G470R}-expressing mutants were statistically assessed to be even greater than those observed in controls heterozygous for murine Smn and WT for Hspa8. This interesting phenomenon was also seen when controls expressing the variant (SMN2; Δ7; Hspa8^{G470R}; Smn^{+/-}) were compared with littermates absent in the modifier (SMN2; Δ7; Smn^{+/-}), and extended to most parameters analyzed, indicating that the modifier potentiates neurotransmission (FIG. 4F). NMJ function in PND8-10 SMA mice, at a time point when all three mouse cohorts were viable, also demonstrated restored neurotransmission in the presence of the modifier. Collectively, this aspect of the study unambiguously assigned a potent SMA-modifying property to the Hspa8^{G470R} protein.

### Hspa8^{G470R} Modestly Raises SMN Levels by Inducing SMN2 Exon 7 Inclusion

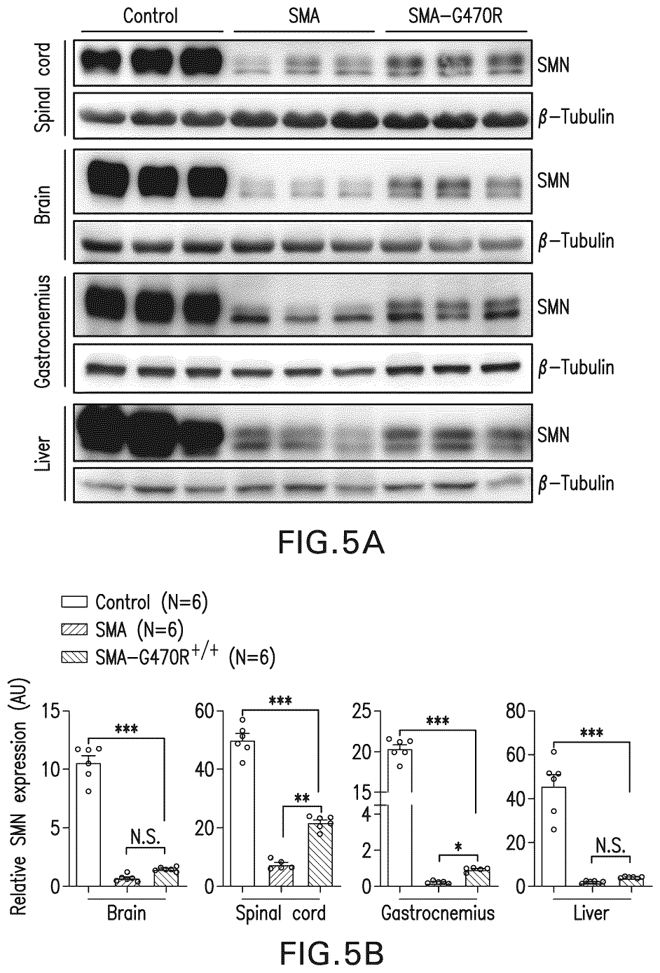

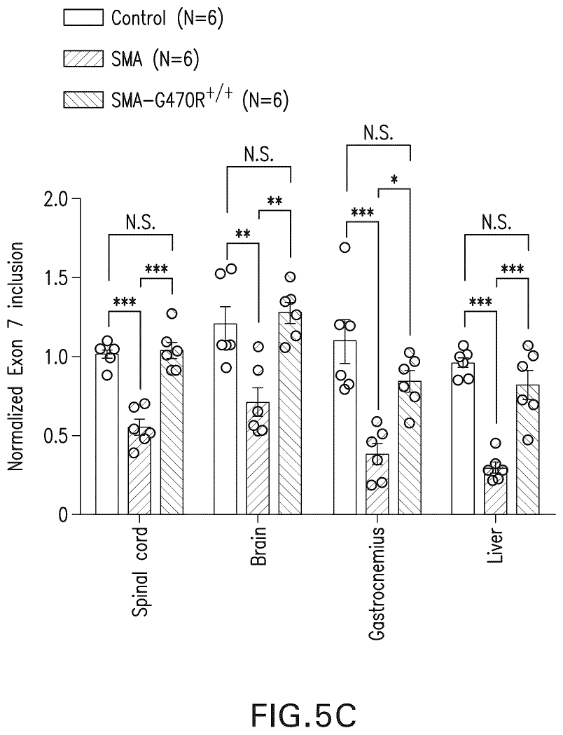

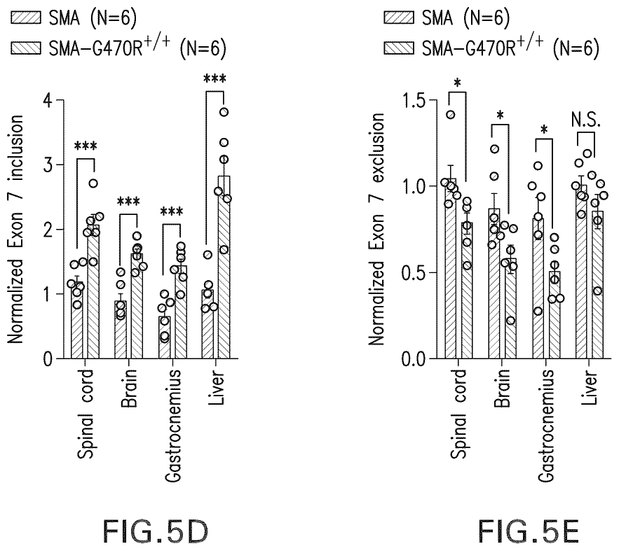

Considering the disease-mitigating effects of Hspa8^{G470R}, we sought to explain its mechanism(s) of action. We began, notwithstanding evidence of acutely low protein in F2 mutants, by again assessing SMN levels. This analysis was undertaken as it had proved challenging, prior to identifying Hspa8^{G470R}, to reliably assort severe and modified mutants into distinct groups for comparison. To our surprise, we found that although modified mutants continue to express only residual protein (≤20%) relative to SMN2^{+/+}; SMN2 Δ7^{+/+}; Smn^{+/-} controls, they nevertheless expressed more SMN than mutants devoid of the modifier (FIGS. 5A and 5B). This suggested that one means by which Hspa8^{G470R} acts is by increasing SMN. Considering the only source of SMN in Δ7 SMA mice is the human SMN2 transgene and that inducing exon 7 inclusion into SMN2 transcripts is one commonly reported means by which MN may be raised, we examined levels of the full-length (FL) and truncated (SMNΔ7) transcripts from SMN2 in our various cohorts of mice by Q-PCR. We found that levels of FL-SMN were indeed modestly enhanced (approximately 2-fold) in modified versus severe mutants (FIGS. 5C and 5D). This result is, moreover, consistent with reports of a feedback loop wherein low SMN protein exacerbates SMN2 exon 7 exclusion, and small increases in protein can stimulate production of FL-SMN transcript.^{27,28} Congruent with an increase in FL-SMN in the modified mutants, levels of SMNΔ7 transcript dropped relative to those in severely affected SMA mice (FIG. 5E). Because measuring these transcripts in Δ7 mice may be confounded by the presence of the SMNΔ7 cDNA transgene¹⁶ in them, we repeated the analysis with mice harboring SMN2 but devoid of SMNΔ7.²⁹ The outcome of the experiments with the latter set of mice reflected results obtained in the Δ7 line of mice; FL-SMN increased, whereas levels of the truncated isoform correspondingly dropped in the presence of Hspa8^{G470R}. This suggested that murine Hspa8^{G470R} raises SMN levels by altering SMN2 splicing.

In a final set of experiments and as measure of the consequences of the Hspa8^{G470R}-dependent increase in SMN expression, we examined the levels of a number of downstream targets of SMN.^{30,31} We found that the reductions in the U12 and U4atac snRNAs seen in severe SMA were reversed and defects of Mdm2 and Clcn7 splicing rescued in modified mutants. However, aberrant splicing of Tmem41b, defective 3′-UTR processing of histone H1c and abnormal Cdkn1a expression persisted in the mildly affected mutants. Unexpectedly, U11 levels were found to increase in severe SMA and perturbed concentrations of this snRNA, U2, and mis-spliced Tspan31 abnormally expressed or actually exacerbated in modified mutants. These results suggest that although Hspa8^{G470R} does raise SMN, the increase is insufficient to fully rescue splicing and/or expression defects universally in genes downstream of SMN.

### Hspa8^{G470R} Exhibits an Enhanced Affinity for its Synaptic Co-Chaperone Partners

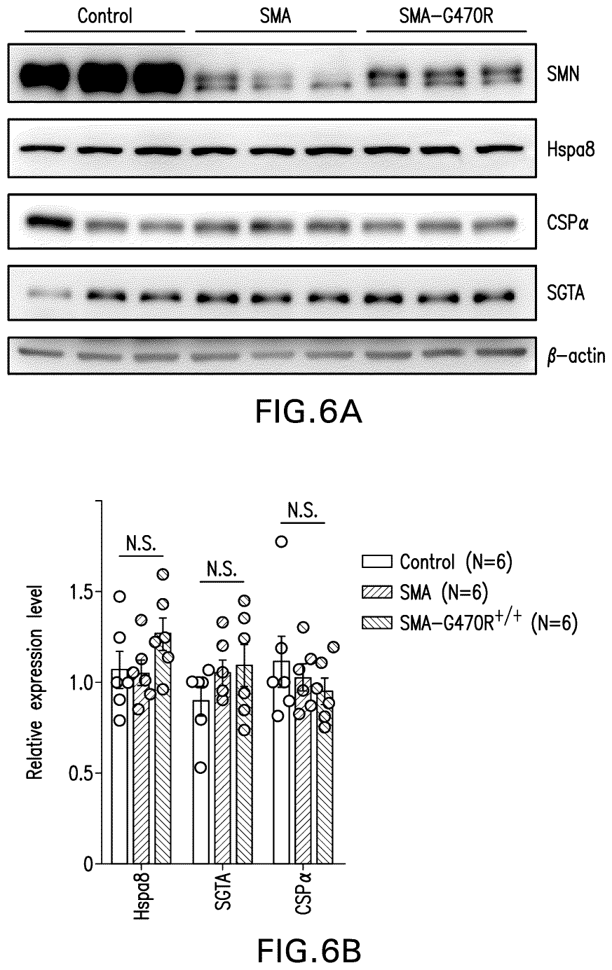

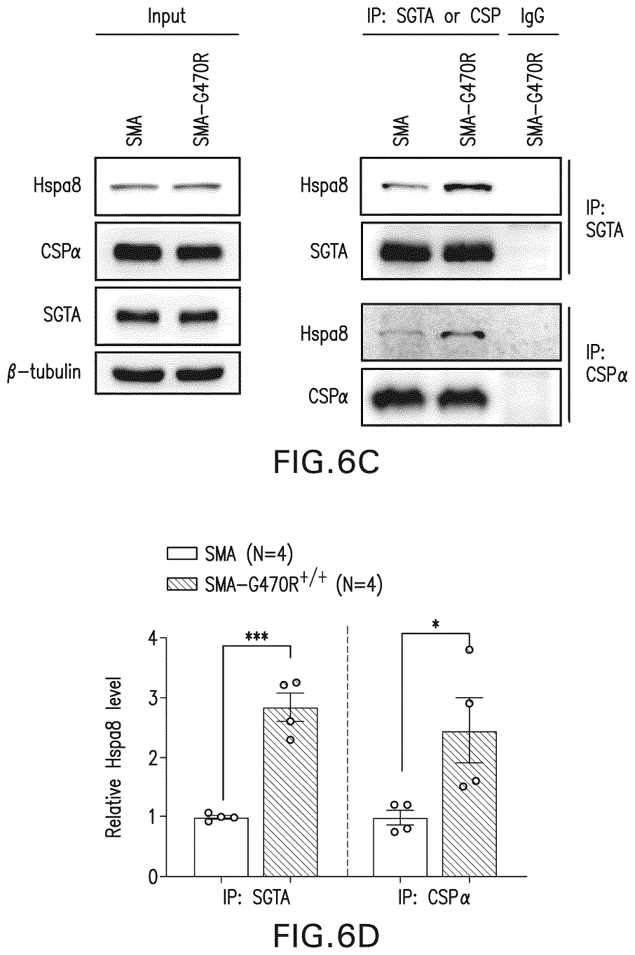

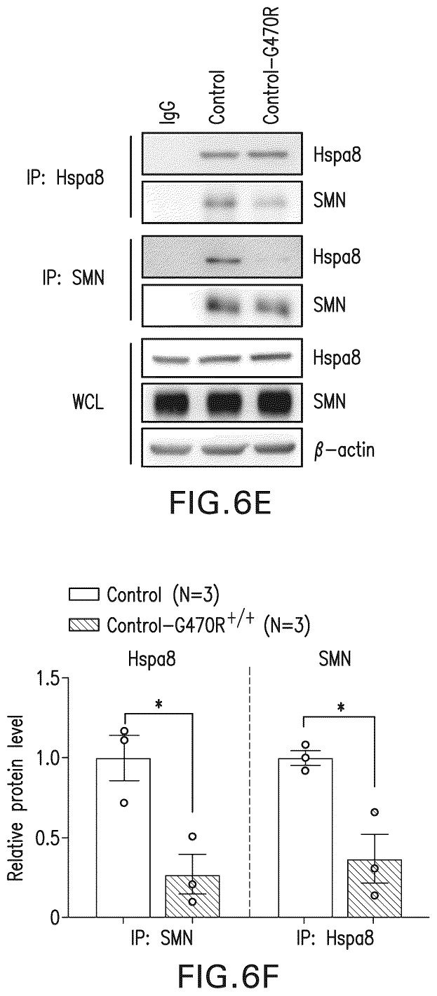

The discordance between the very modest increase in SMN and the striking rescue of the SMA phenotypes in our mutants led us to speculate that Hspa8^{G470R} modifies disease directly and in addition to its effects on SMN. This idea was especially appealing considering the many synaptic functions of Hspa8^{32,33} and our own observations suggesting a potentiation of neurotransmission by Hspa8^{G470R}. Indeed, Hspa8 is an important member of a tripartite chaperone complex critical for proteostasis and recycling of a number of synaptic proteins including those of the SNARE complex involved in neurotransmission.^{34,35} We, therefore, inquired if the other two members, SGTA and CSPa, of the tripartite chaperone of which Hspa8 is a component were affected under conditions of low SMN or by the Hspa8^{G470R} variant. We found that levels of SGTA and CSPα were unaffected by low SMN (FIGS. 6A and 6B). Yet, interestingly, the G470R variant enhanced the affinity of Hspa8 for its binding partners (FIGS. 6C and 6D) that the strengthened interaction was not just SMN-mediated, given the incremental increase in SMN in modified versus severe mutants, and was confirmed by extending the analysis to control samples. A large increase in SMN in these control samples, WT for Hspa8, did not augment the interaction of the latter for SGTA to the extent discerned in mutants harboring the variant. Congruently, Hspa8^{WT}-SGTA binding remained weaker than the binding of the G470R variant to SGTA in Smn heterozygotes. We also discovered that Hspa8 is an SMN-interacting partner, and this association was significantly weakened by the variant (FIGS. 6E and 6F). We suggest that binding of Hspa8 to the two other members of the tripartite chaperone complex occurs while potentially being competed for by other client protein interactors such as SMN. These interactions, which likely influence one another and have downstream functional consequences, are altered by Hspa8^{G470R}.

### SNARE Complex Assembly is Disrupted by Low SMN and Recovers in SMA; Hspa8^{G470R} Mice

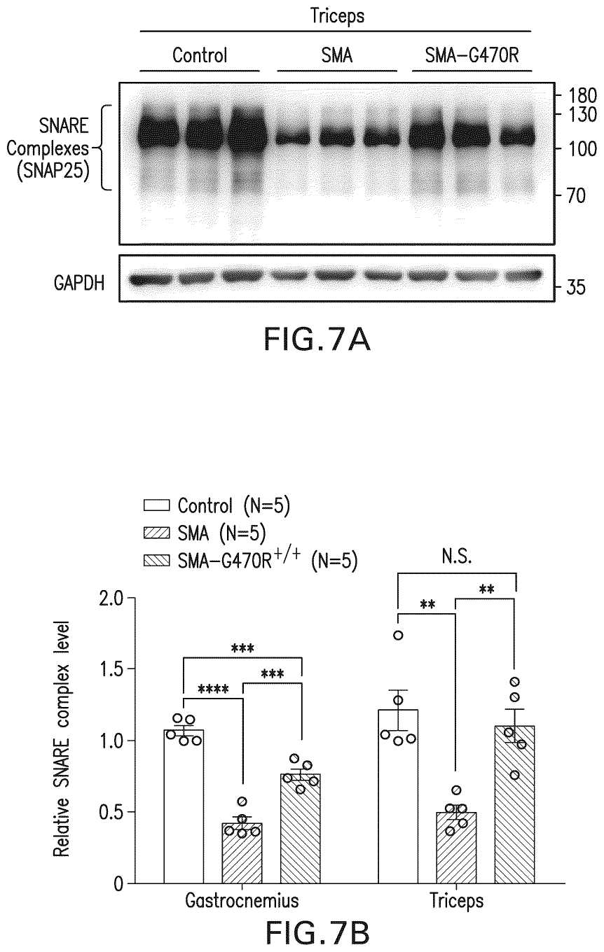

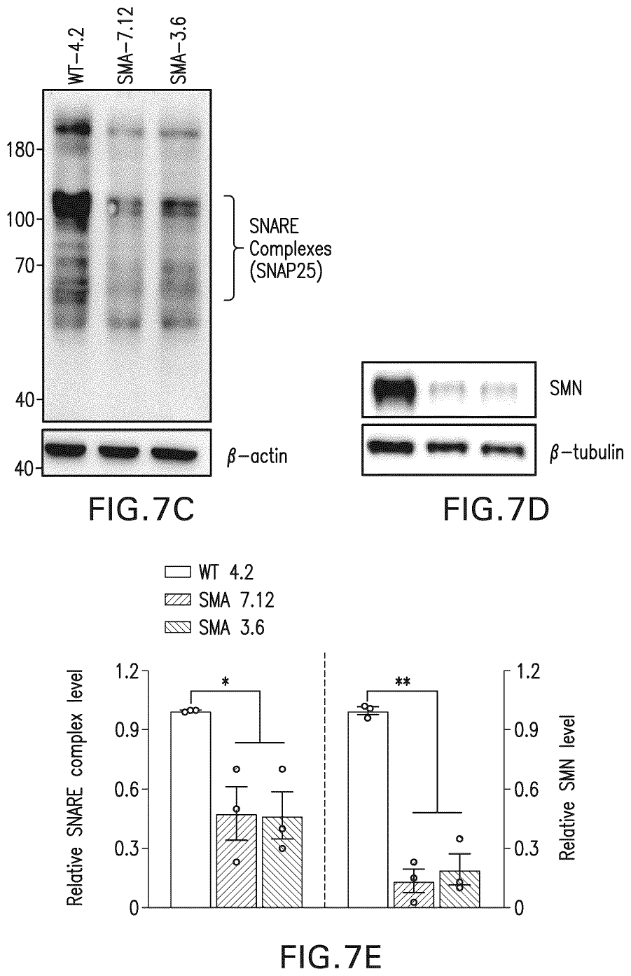

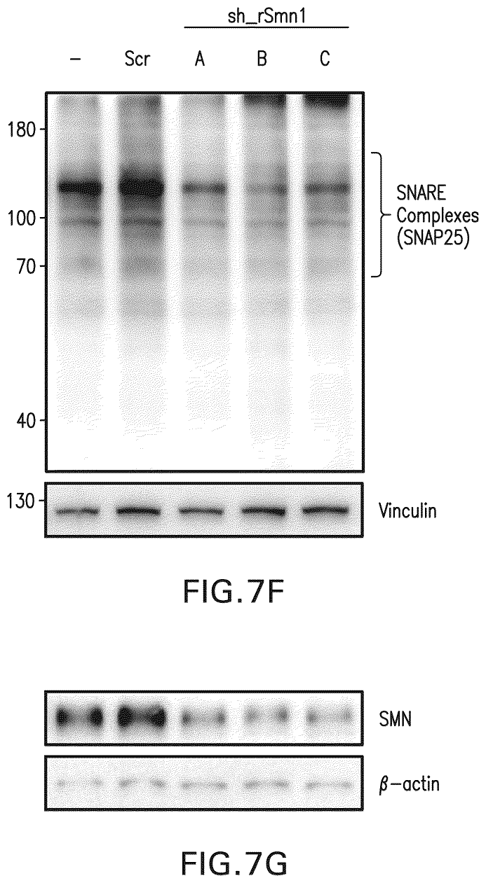

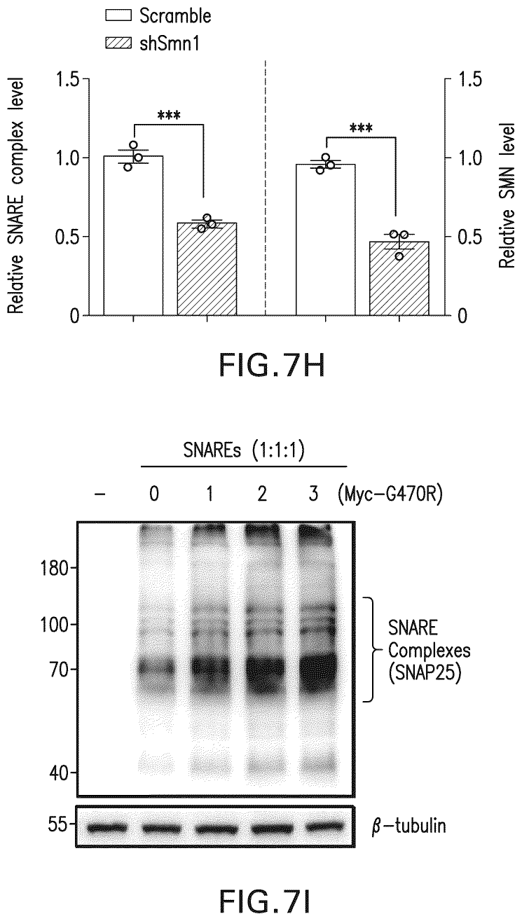

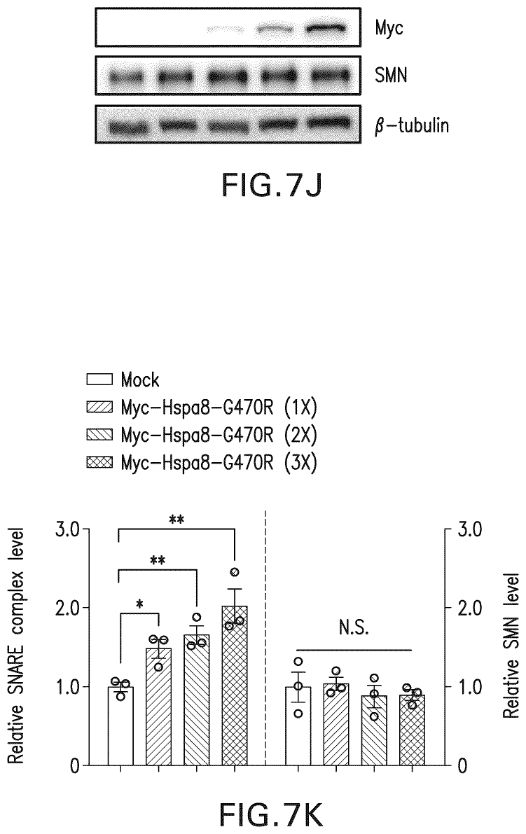

Considering the implications of proper chaperone complex formation and function for SNARE complex assembly, and the upshot-poor neurotransmission-of inefficient SNARE complex formation, we quantified assembled SNARE complexes in modified and severe mutants. We turned to muscle (triceps) tissue, a source of neuromuscular synapses; these synapses are particularly vulnerable in SMA. Interestingly, this analysis revealed a clear and reproducible difference between severe SMA mice and healthy littermate controls heterozygous for murine Smn; assembled SNARE complexes as detected with an antibody against SNAP25, a core component of the complex, were significantly less abundant under conditions of low SMN, but absent in any attendant drop in levels of SNAP25, Stx1, or Syb2 monomers. Importantly, their levels were restored in modified mutants, and this pattern observed, albeit to a lesser extent, in gastrocnemius muscle (FIGS. 7A, 7B).

Given the novelty of SNARE complex assembly deficits in SMA, we sought to investigate underlying mechanisms. Accordingly, we first repeated assessments of assembled SNARE complexes, probing our blots with antibodies against the two remaining components, Stx1 and Syb2, of the complex. These experiments also revealed inefficient SNARE complex assembly in severe SMA and rescue of the assembly in Hspa8^{G470R}-expressing mutants. Next, we examined SNARE complex assembly in iPSC-derived motor neurons from severe SMA patients. This too revealed fewer assembled complexes under low SMN conditions (FIGS. 7C-7E). The lower abundance of SNARE complexes detected in the iPSC-derived motor neurons with the SNAP25 antibody were confirmed with antibodies against Stx1 and Syb2. Similarly, knockdown of SMN in neuron-like PC-12 cells resulted in inefficient SNARE complex assembly (FIGS. 7F-7H). Third, we ascertained the likelihood that rescue of SNARE complex assembly in modified mutants was mediated exclusively through induction of SMN by Hspa8^{G470R} or, alternatively, at least in part through a direct effect of the variant chaperone on the SNARE complex. For this, we examined complex assembly in two sets of Smn^{+/-} controls expected to assemble sufficient complexes for normal neurotransmission. Yet, controls expressing the Hspa8^{G470R} variant were found to assemble SNARE complexes more efficiently than cohorts harboring WT Hspa8. This suggested that Hspa8^{G470R} promoted SNARE complex assembly even under non-disease-relevant SMN conditions and without a requirement for large increases in SMN. Indeed, we found that over-expressing Hspa8^{G470R} in HEK293 cells transfected to express equimolar quantities of Stx1, Syb2, and SNAP25 resulted in correspondingly greater levels of SNARE complexes (FIGS. 7I-7K). Interestingly, in these experiments, an appreciable increase in SMN was not detected (FIGS. 7J and 7K) indicative of a direct effect of Hspa8^{G470R} on SNARE complex formation. Moreover, forced expression of SMN in HEK293 cells beyond WT levels failed to stimulate SNARE complex assembly the way Hspa8^{G470R} did. This suggested that Hspa8^{G470R} has a direct and potent effect of potentiating SNARE complex assembly. In the context of fewer such complexes forming in SMA NMJs, Hspa8^{G470R} likely restores their levels, rescuing neurotransmission at these synapses. In aggregate, we posit that Hspa8^{G470R} has a significantly more robust and direct effect on restoring SNARE complex assembly in our mutants instead of merely acting to promote the formation of these complexes by raising SMN. Notwithstanding such an inference, and given the clear dependency of SNARE complex assembly on SMN levels, we performed a fourth set of experiments to inquire if poor complex formation was a peculiarity of just the Δ7 line or more generally observed in SMA. To do so, we examined complex formation in a second SMA mouse model—the Smn^{2B/-} mouse.³⁶ Poor SNARE complex assembly was determined to be a feature of this model too, suggesting that this phenomenon is a likely bona fide synaptic defect in SMA. In a final set of experiments, we inquired if inefficient SNARE complex formation in SMA mutants is a mere byproduct of denervation by examining how well the complexes formed in a mouse model of amyotrophic lateral sclerosis (ALS).³⁷ We found that complex assembly in symptomatic (PND110-127) ALS mutants was no different from that in controls. Indeed, in this particular mutant SOD-1 line expressing Hspa8^{G470R} failed to modify the ALS phenotype. In aggregate, the data reveal a new aspect of SMN function—a capacity to modulate, likely indirectly, SNARE complex assembly. The data also suggest that Hspa8^{G470R} functions in the same pathway to independently promote complex formation and, finally, that inefficient formation of this synaptic complex is not merely a general consequence of a denervating disease.

### DISCUSSION

The durability of current SMA treatments remains unclear, and the precise function(s) of the SMN protein in maintaining the vigor of the neuromuscular system continues to be debated.³⁸ Here, we report findings that inform how nerve-muscle function is preserved by proteins best recognized for their housekeeping properties. Morphological and functional defects in model mice expressing Hspa8^{G470R}, a novel and potent SMA suppressor, were rescued, motor neuron numbers restored, and lifespan enhanced from approximately 10 days to roughly 300 days. Such a profound rescue has not previously been reported for severe SMA. Our finding emerges from functions attributed to Hspa8, implicating SMN in a novel role consistent with the classic, neuromuscular SMA phenotype—influencing SNARE complex assembly at nerve-muscle synapses. Building on the discovery that the G470R variant is better able than WT Hspa8 to bind other constituents of a tripartite synaptic chaperone critically important for SNARE complex formation, we found that assembly of this complex is disrupted at NMJs and spinal motor neurons respectively of severely affected SMA mice and humans. In contrast, SNARE complex formation was restored in the presence of the modifier; NMJ function was concomitantly normalized, indeed potentiated, in Hspa8^{G470R}-expressing SMA mutants. These findings cast new light on how low SMN triggers motor neuron dysfunction. Aside from a handful of human studies that identified perturbations of endocytosis in SMA,¹²⁻¹⁵ positional cloning strategies and unbiased genetic screens to probe disease mechanisms have not been particularly informative for this condition. Moreover, none of the investigations employed humanized SMA model mice. Here, we describe the outcome of one such exploration for SMA modifiers using the rodent models. Wild-type Hspa8 is an especially abundant component of synapses³⁹ and best known for ensuring cellular homeostasis through the sequestration or degradation of nascent, misfolded or aggregated proteins.⁴⁰ However, it is in two lesser-known capacities—as a chaperone of neuronal SNARE complexes and as a modulator of splicing that the G470R variant most likely alters the SMA phenotype. Hspa8 is not widely reported to be involved in splicing. Still, it has been detected by mass spectrometry analysis in mammalian spliceosome complexes and, additionally, emerged in an unbiased screen for splice modulators as a weak activator of PSD-95 splicing.⁴¹⁻⁴³ WT Hspa8 may be a constituent of the factors that regulate SMN2 splicing, either directly as a member of the spliccosome or, indirectly, by altering an SMN2 splicing protein in its capacity as a chaperone. Substituting Gly470 in Hspa8 with Arg likely alters the levels and/or activities of these factors resulting in exon 7 inclusion in the SMN2 transcript. The substitution concomitantly weakens Hspa8-SMN interaction but enhances the affinity of Hspa8 for its binding partners in the tripartite chaperone complex, likely increasing the efficiency with which it assembles into a functional unit to promote SNARE complex formation.

We suggest that the disrupted SNARE complex assembly in SMA results, at least in part, via unintended competition between client proteins such as SMN and SGTA/CSPα for Hspa8, entrapment of Hspa8 into SMN-containing units and, consequently, inadequate chaperone complex to maintain synaptic SNAREs in proper conformation to sustain repeated cycles of neurotransmission.

The Hspa8^{G470R} SMA suppressor we report here has emerged from observations of discordant phenotypes in humanized model mice and the most potent reported so far. This likely stems from multiple mechanisms of action. Our study provides evidence for involvement of at least two pathways, one based on increased SMN from the human SMN2 gene, the other most likely acting directly on neurotransmission via effects on SNARE complex assembly. The relative contributions of the two pathways identified here to overall disease mitigation remain to be determined, as small changes in SMN can significantly alter disease.⁵⁹⁻⁶¹ The net effect observed here was a complete rescue of neuromuscular dysfunction-as assessed in young adult mutant mice—but failure to resolve defects of splicing fully. This bolsters the notion that the neuromuscular SMA phenotype is not immutably linked to defects of splicing.

### METHOD DETAILS

### Animals

Δ7 SMA model mice (Jax #005025), and their progenitors harboring only human SMN2 (Jax #005024), were created by us^{16,29} and are available from the Jackson Labs. Transfer of the various transgenic and knockout alleles constituting the Δ7 model onto the FVB/N and C57BL/6 strain backgrounds was carried out at Columbia University. SOD^{G86R} mutants were acquired from the Jackson Labs (#05110). Hspa8^{G470R} mice were created at the Columbia University mouse core facility using CRISPR technology. To generate Hspa8^{G470R} mice, a donor template (5′-TATGAAGGTGAAAGGGC CATGACCAAGGACAACAAC CTGCTTGGAAAGTTCGAGCTCACAGGCATCCCTCCAGCACCCCGTCGGGTCCCTC AGATTGAGGTTACTTTTGACATCGATGCCAATGGCATCCTCAATGTTTCTGCTGTA GATAAGAGCACA-3′; SEQ ID NO:2) and Hspa8-G470R guide RNA (sgRNA: 5′-GCAUCCCUCCAGC ACCCCGGUUUUAGAGCUAUGCU-3′; SEQ ID NO:3), were synthesized (Integrated DNA Technologies). S. pyogenes CAS9 nuclease was purchased (New England Biolabs). Equimolar concentrations of CAS9 enzyme and sgRNA were mixed in injection buffer (10 mMTris-HCl, pH 8.0, 0.1 mMEDTA, pH 8.0) to form 0.15 mMRNP complex. The RNP complex (0.15 mM) and the donor template (0.5 mM) were injected into the pro-nuclei of fertilized FVB/NJ eggs and transferred after a 24 h incubation period into the oviducts of pseudo-pregnant surrogate females. Analyses of the various mice employed for the study were carried out in a blinded fashion except for behavioral studies when severe phenotypes displayed by mutants precluded blinding. All animal procedures adhered to protocols described in the Guide for the Care and Use of Lab Animals (National Academies Press, 2011). The subjects of this study were randomly selected, mixed or pure background male and female mice housed in a controlled environment on a 12-hour light/12-hour dark cycle with food and water. Δ7 SMA carrier mice heterozygous for murine Smn constituted healthy controls unless otherwise specified. Genotyping was performed by PCR on tail DNA. Righting ability was assessed as previously described.²⁰ Briefly, mice were placed on their backs and latency of the animals to place all four limbs on the tabletop recorded. Time to right was converted into a score as described²⁰ and reported as such in the manuscript. For the behavioral studies, GraphPad Prism was used to determine sample sizes to detect differences of at least 2 SDs with a power of 80% (P<0.05). Mice were not randomized, but in any instance where mutants did not exhibit an overt disease phenotype, it was possible to blind the investigator to the particular cohort being assessed.

### Sequencing and GWAS

Genomic DNA from 25 F2 SMA mutants exhibiting either severe or mild disease was isolated and subjected to whole genome sequencing using the NovaSeq 6000 platform (Novogene Inc.). An average of 52 billion bases per sample were sequenced and roughly 333 million reads per subject mapped by aligning the reads to the C57BL/6 reference sequence (GRCm38.p6/mm10) using the Burrows-Wheeler Aligner algorithm.⁶² Removal of duplication reads from the BAM files thus generated resulted in sequence coverage of 11X-14X (>95% of genomic regions covered at a depth of 4X and 98% of the genome covered at a depth of 1X). Samtools⁶³ and ANNOVAR⁶⁴ were used to call SNPs from BAM files and to predict the consequences of the variants, respectively. Genome-wide association tests were performed with the following phenotypic groups stratified based on increasing degree of disease severity (LL1-11, I1-8, and S9-14; LL: long-lived, I: Intermediate; S: Severe) and assigned numeric values; LL1-11=1, I1-8=2, S9-14=3. Tests were run under linear models by using the-glm function in PLINK2 software (https://www.cog-genomics.org/plink/2.0/). ANNOVAR⁶⁴ was used to predict consequences of the 4742 significant SNPs, P values<5×10⁻⁸.

### Motor Neuron, NMJ and Muscle Histology

Motor neuron, NMJ and muscle histology was essentially carried out as previously described^{20,65} and detailed below. Motor neurons: Spinal cord tissue was dissected following transcardial perfusion (4% PFA in 1X PBS) of mice. The tissue was post-fixed in the same fixative, cryo-protected in 20% and then 30% sucrose before embedding the material in Tissue-Tek OCT medium for cryostat sections. 20 μm sections from lumbar spinal cords were overlaid for 10 minutes with 4% PFA, washed in 1X PBS and the tissue then permeabilized with 1% Triton X-100, 5 min. To stain the motor neurons, sections were placed in blocking buffer (2% normal goat serum, 2% BSA, 0.5% Triton X-100 in PBS) for 1 hr at room temperature. The sections were then incubated (4° C., overnight) with primary antibodies against ChAT (1:100, Millipore) or vGlut1 (1:10,000, Millipore) diluted in blocking buffer, and washed (15 min×4) with 1X PBS. Sections were subsequently incubated with appropriate secondary antibodies (Alexa Fluor-594 conjugated donkey anti-goat IgG or Alexa Fluor-488 conjugated goat anti-guinea pig IgG, Invitrogen) at dilutions of 1:1000, 2 hr at room temperature. After washing (15 min×4) with 1X PBS, the sections were mounted in anti-fade mounting media (Vector Labs) and motor neurons visualized on either a Nikon 80i fluorescent microscope (Nikon) or a Leica TCS SP5 II laser scanning confocal microscope (Leica). Motor neuron numbers were assessed in the L1-L5 region of the spinal cord and the raw numbers extrapolated to the entire region based on the length and thickness of tissue segment.

### Muscle Histology

The proximal triceps muscle and distal gastrocnemius muscle were flash-frozen in isopentane cooled with liquid nitrogen. 12 μm thick sections were stained with hematoxylin and eosin (H&E; Sigma), and morphology, size and numbers of fibers were determined using ImageJ software following image acquisition with a SPOT 4.5 camera and associated software (Diagnostic Instruments). Fiber sizes were determined by assessingR 100 fibers from individual samples. Cardiac structure was assessed using transverse sections cut at the level of the ventricles. The thickness of the left ventricles in mutants was normalized to heart size.

### NMJ Assessment

NMJ analysis was performed on whole muscle. Tissues were fixed and permeabilized with 100% methanol for 10 min at −20° C. and incubated with blocking buffer (2% normal goat serum, 3% BSA, 1% Triton X-100 in PBS) for 1 hr at room temperature. The tissue was incubated overnight at 4° C. with a primary antibody against neurofilament (NF, 1:1000, Millipore) and washed (20 min×4) by 1X PBS. The following secondary antibodies were subsequently applied: Alexa Fluor-488 conjugated goat anti-rabbit IgG secondary antibody (1:1000, Invitrogen), and rhodamine-a-bungarotoxin (BTX, 1:1000, Invitrogen) for 3 hr at room temperature. After washing (20 min×4) by 1X PBS, the tissue was mounted in anti-fade medium (Vector Labs) and images of NMJs were acquired with Leica TCS SP5 II laser scanning confocal microscope (Leica). Morphological analyses were conducted using LAS-X software (Leica). NMJ abnormalities were quantified as previously described.²⁰

### NMJ Electrophysiology

NMJ function was assessed by electrophysiological means as described by us in prior reports.^{21,66} Detailed protocols follow. Methods described in a previous report⁶⁶ were used to evaluate NMJ function in modified F2 SMA mutants. In short, EDL muscle with the sciatic nerve attached was extracted from 4-5 month-old mice and placed in oxygenated mammalian Ringer's solution. Muscle contraction was suppressed by incubating the tissue (45 min) in 2-3 mm m-Conotoxin. Recordings were subsequently carried out in toxin-free Ringer's solution. Between 40 and 70 MEPPs and 20 EPPs were gathered from each NMJ. EPPs were triggered with a 1Hz train, normalized to −75 mV and corrected for non-linear summation. Mean quantal content was calculated from the ratio of EPPs to MEPPs. The protocol for assessing NMJs in SMA-G470R+/+ mutants and controls differed somewhat and is detailed in a previous report.²¹ Briefly, TA (tibialis anterior) muscle with the intact nerve was dissected from mice at ˜PND75 and placed in oxygenated mammalian Ringer's solution. NMJs were visualized with 10 mm 4-(4-diethylaminostyryl)-N-methylpyridinium iodide (4-Di-2ASP), then impaled at a distance no greater than 100 mm from the endplate. Myofibers were crushed away from the endplate band and voltage clamped to −45 mV to prevent movement after nerve stimulation. Two-electrode voltage clamp was employed to determine miniature endplate current (MEPC) amplitudes and endplate currents (EPCs) evoked after nerve stimulation. Voltage clamp precludes issues stemming from differences in muscle fiber size, e.g., differences in capacitance and input resistance. Quantal content was calculated by dividing EPC amplitude by the mean MEPC amplitude for a given NMJ. NMJ function in PND8-10 mice was assessed in the TVA muscle as described previously.

### Cardiovascular Function

Cardiac function was evaluated according to protocols previously employed by us.^{68,69} Briefly, mice were lightly anesthetized (1-2% isoflurane) before performing transthoracic echocardiography using a Visualsonics Vevo 770 ultrasound system (Visualsonics) with a 30 MHz transducer applied to the chest wall. M-mode images and two-dimensional (2D) parasternal short-axis images at the mid-papillary muscle level were recorded. Measurements were made offline by a single individual in a group-blinded fashion. Assessments of left ventricular (LV) end-diastolic and end-systolic internal diameters were made enabling calculation of LV fractional shortening (FS), LV end-diastolic volume, LV end-systolic volume, and ejection fraction (EF). End-diastolic and end-systolic cavity areas were quantified at the mid-papillary level by tracing the endocardial border. The % LV fractional area change was estimated using the formula: [(LV end-diastolic cavity areas−LV end-systolic cavity areas)/LV end-diastolic cavity areas]×100. M-mode images were used to determine heart rates. All parameters represent the mean of three beats.

### Transcript and Protein Measurements

Total RNA was extracted using TRIzol reagent (Invitrogen) according to the manufacturer's instructions and then treated with RNase-free DNase I (Thermofisher). cDNA was synthesized using the RevertAid RT kit (Thermo) or the iScript Reverse Transcription Supermix (Bio-Rad). Quantitative PCR was performed in triplicate on a CFX96 Real-Time System (Bio-Rad) using SsoAdvanced Universal SYBR Green Supermix (Bio-Rad), and normalized to endogenous Gapdh or b-Actin mRNA levels. For protein estimation, western blot analysis was performed. Protein was extracted from cells or tissues using lysis buffer (50 mM Tris-HCl pH 7.5, 150 mM NaCl, 1 mM EDTA, 1% NP-40, protease Inhibitor Cocktail; Roche-#4693159001). In the case of tissue-derived protein, tissue was homogenized and incubated in ice for 30 min. Lysates were cleared by centrifugation (15,000 g for 30 min at 4° C.) and then protein levels quantified using the BCA protein assay kit (Thermo). Prior to loading protein samples on SDS gels for immunoblotting, lysates were denatured by boiling for 5 min with 4X Laemmli sample buffer (Bio-Rad), or were mixed with 2X Lacmmli sample buffer (Bio-Rad)—without boiling—to measure SDS-resistant SNARE complexes. To detect the lower abundance of SNARE complexes in the hiPSC lines, the protocol was slightly modified such that the lysis buffer indicated above was replaced with 1X PBS. For co-IP studies, 0.5-1 mg of protein lysate was incubated (4° C., overnight) with primary antibodies, then 20 ml of protein A/G PLUS-Agarose beads (SantaCruz) added to the lysate and the mix further incubated (4° C. for 4-6 hr). The beads were washed 5 times with lysis buffer, and bound proteins eluted in 2X Laemmli sample buffer (Bio-Rad) before performing SDS-PAGE and immunoblotting. Separated proteins were transferred to PVDF membrane (Millipore), membranes blocked in TBS containing 5% non-fat milk and 0.1% Tween 20, and primary antibodies used to probe (2 hr at room temperature or overnight at 4°° C.) for the presence of relevant proteins. Following incubation with primary antibodies, membranes were washed (1X TBST) and then incubated (1 hr, room temperature) with appropriate secondary antibodies. Protein bands were visualized using the ECL kit (Bio-Rad) and images captured with a ChemiDoc Imaging System (Bio-Rad). Band intensities were determined with the Image Lab (Bio-Rad) or ImageJ software.

### Cell Cultures and Transfections

PC-12 cells (ATCC CRL-1721) for our experiments were maintained in RPMI-1640 supplemented with 10% horse serum and 5% fetal bovine serum (FBS); HEK293T cells were maintained in DMEM supplemented with 10% FBS at 37° C. with 5% CO2. Knockdown of SMN in the PC-12 cells was accomplished using lentivirus expressing a rat Smn shRNA. Lentivirus was produced by co-transfecting the following three plasmids into HEK293 cells: pGFP-C-shSmnllenti (Origene, #TL710710), the pCMV-D8.2 packaging vector (Addgene, #12263) and pMD2.G (Addgene #12259), which codes for the virus envelope. Culture media from the transfected cells was collected 48 h and 72 h post-transfection, combined and viral particles in the liquid concentrated using the Lenti-XTM Concentrator (Clontech) according to the manufacturer's instruction. To assess SNARE-complex assembly in HEK293 cells, a plasmid expressing rat Syntaxin 1A, VAMP2 and SNAP25 (1:1:1) under the control of the CMV promoter was transiently co-transfected into the cells (Lipofectamine 3000, Invitrogen) along with a construct containing Myc-tagged mouse Hspa8-G470R or human SMN1. Forty-eight hours later, the cells were harvested, lysed and protein extracted for immunoblotting. SMA iPS cells were generated and maintained on irradiated mouse embryonic fibroblasts. Motor neurons from the iPS cells were derived from human induced pluripotent stem cell (hiPSC) lines from 1× healthy individual (line 4.2), 1× SMA Type 1 patient (line 7.12) and 1× SMA Type 2 patient (line 3.6; previously classified as Type 1). hiPSCs were differentiated into spinal cord-like motor neurons using a previously established protocol.^{70,71} iPSCs were maintained on Corning® Matrigel® Growth Factor Reduced Basement Membrane Matrix coated 6-well plates in Essential 8 medium. Neurospheres were generated from iPSCs via dual SMAD inhibition (SB 431542 and LDN 1931899) and Wnt activation (Chir 99021) and subsequently patterned with retinoic acid and smoothened agonist to induce a ventral-caudal cell fate. Spinal motor neuron progenitors were dissociated from neurospheres and plated down on to Matrigel_ coated plates with maturation media supplemented with DAPT, BDNF and GDNF for terminal differentiation. Cells were cultured to Day 50, collected and snap frozen for protein extraction.

### Quantification and Statistical Analysis

Kaplan-Meier survival curves were assessed for differences using the log-rank test equivalent to the Mantel-Haenszel test. The unpaired, 2-tailed Student's t test, one-way ANOVA followed by Tukey's post-hoc comparison or Kruskal-Wallis test followed by Tukey's or Dunn's post-hoc comparison, where indicated, were used to compare means for statistical differences. In instances where categorical binomial variables had to be analyzed, the Fisher's exact test was employed. Data are represented as mean±SEM unless otherwise indicated. P<0.05 was considered significant. Statistical analyses were performed with GraphPad Prism v6.0 (GraphPad Software).

### Example 2

A signature feature of the age-related neurodegenerative dementias that include some forms of Alzheimer's disease (AD), frontotemporal dementia linked to chromosome 17 (FTDP-17) and progressive supranuclear palsy (PSP) is the appearance of tau-containing neurofibrillary tangles (NFTs) within brain neurons. The present method may be used to treat tau-related dementias. The extent of NFT pathology correlates with disease progression. Moreover, such pathology in a subset of FTDP-17 patients with underlying tau mutations suggests that mutant tau is sufficient to cause tangle formation and trigger neurodegeneration. Although the link between tau aggregates and neurodegeneration has been recognized for decades, little has been achieved in exploiting this link to treat AD and other tauopathies.

Studies have shown that tau homeostasis is critically dependent on a network of cellular chaperones, most notably Hspa8, an abundant and constitutively expressed member of the Hsp70 family. When dissociated from microtubules, particularly following changes in its conformation, tau is capable of binding Hspa8. Under normal circumstances Hspa8 either restores tau to its properly folded structure, releasing it for subsequent rounds of microtubule assembly or directs it to the proteasome for degradation. However, when tau loses its function and gains a toxic modification such as aberrant phosphorylation, Hspa8 fails to release it thus inadvertently contributing to preservation of the abnormal tau and promoting aggregate formation. Inhibiting Hspa8 or subtly altering its substrate binding domain through which it interacts with tau could preclude this situation. The first of these strategies has indeed been investigated and shown to increase tau turnover suggesting one therapeutic means of preventing NFT pathology in tauopathics.

We have shown that a G470R variant in Hspa8 potently suppresses disease in a monogenic neurodegenerative disorder—spinal muscular atrophy. Importantly, part of the suppression involved preventing the characteristic aggregation of neurofilaments within neuromuscular junctions (NMJs) of SMA model mice. It is expected that Hspa8^{G470R} will similarly combat abnormal tau aggregation. Our knock-in Hspa8^{G470R} mice have displayed little evidence of disease even when homozygous for the variant. This suggests that G470R does not generally disrupt proteostasis in the intact organism.

The present method can target a molecular chaperone as a means to enhance turnover of abnormal tau. Hspa8 null alleles are lethal. The present Hspa8 variant, which lies within the substrate binding domain of the chaperone, is expected to reduce the affinity of Hspa8 for client proteins such as mutant tau and accordingly enhance turnover. The present method can suppress tau by modulating the chaperone network in an intact organism. For example, this may be assessed in mice constitutively expressing the Hspa8^{G470R} variant and therefore in the pre-symptomatic animal. Mutants will also be examined following symptom onset. The present method may employ a viral vector to deliver the therapeutic chaperone for the treatment of AD and related tauopathies. The vectors may be AAV-PHP.cB, which can transduces the CNS well, and AAV9.

To determine the cellular basis of the phenotypic rescue by Hspa8^{G470R}, we examined muscle nerves and neuromuscular junctions (NMJs) of PND7 SMA mutants with or without the disease suppressor. A characteristic feature of SMA in patients and model mice is the abnormal accumulation of neurofilament (NF) protein in muscle nerve and nerve terminals. We found that this pathology had been completely ameliorated in SMA model mice harboring the Hspa8^{G470R} variant (FIGS. 4A-4D). NF like tau is an Hspa8 client protein and subject to Hspa8-mediated proteasomal degradation. Considering the effect of Hspa8^{G470R} on NF aggregates in SMA, the variant is expected to have a similar effect on abnormal accumulations of tau in AD and related tauopathies. We assessed NMJ function electrophysiologically as a means of determining the physiological effects of Hspa8^{G470R} on the SMA phenotype. Consistent with the overall phenotypic rescue, we found that quantal content was restored to normal in SMA mutants harboring Hspa8^{G470R}; SMA mutants WT for Hspa8 exhibit profound neurotransmission defects including reduced quantal content. The present method of treating tau-related diseases may be through the suppression by Hspa8^{G470R} of NF aggregates in muscle nerves and at NMJs.

### Experiments

There is a direct correlation between the accumulation of tau into neurofibrillary tangles and cognitive decline in individuals afflicted with AD and related dementias. As of yet there is little to halt the inexorable deterioration of these patients. Arresting tau pathology can be used for treatment. This may be achieved by targeting the family of Hsp70 chaperones. Indeed, modulating the expression of these chaperones promoted tau degradation. Selectively targeting the most abundantly expressed member of this family, Hspa8, is expected to have the similar/same effect. We have demonstrated that a variant of Hspa8 has a striking disease-modifying effect on the SMA phenotype, e.g., by ameliorating NF aggregation in neuromuscular synapses. We expect that the variant has a similar effect in the context of tau pathology.

### Experiment 1—Biochemical Interaction and Turnover of tau in the Presence of Hspa8^{G470R}

We will examine if and to what extent the G470R variant in Hspa8 affects its interaction with and turnover of the tau protein. To do so we will exploit mice with or without the G470R knock-in; the knock-in mice have been generated. Tau will be immuno-precipitated from brain extracts of mice either homozygous for Hspa8^{G470R} or WT for the chaperone and levels of Hspa8 bound to tau assessed in each case by western blot analysis. We will complement this study using primary cultures of cortical neurons which enable treatment of the neurons with microtubule de-stabilizers such as nocodazole; destabilization of microtubules is known to cause dissociation of tau from the microtubules and enhance Hspa8-tau interaction. We expect that the G470R variant in Hspa8 will reduce the affinity of the chaperone for tau.

In parallel with the binding studies, we will quantify total levels of tau in brain extracts and cultured neurons from mice with or without Hspa8^{G470R}. As in the binding experiments, we will consider examining tau levels in brain neurons in the presence of nocodazole. We expect that tau levels will be decreased in tissue extracts and neurons from mice expressing the Hspa8^{G470R} variant.

### Experiment 2—Does Genetic Expression of the Hspa8^{G470R} Variant Mitigate Disease in the rTg4510 Model of AD?

To test the effects of Hspa8^{G470R} tau pathology, we will employ the repressible rTg(tau_{P301L}) 4510 line of mice. This well-established model of tau-associated dementia exhibits significant tau pathology as well as impaired memory and learning when the inducible tau_{P301L} transgene is activated in brain tissue. Also known as rTg4510, the model relies on intercrossing 1) a “responder” line (Jackson Labs—stock #015815) in which the tau transgene is placed in the mouse prion locus under the regulatory control of a tetracycline response element (TRE) and 2) an “activator” line e.g., CaMKIIa-tTA in which a tetracycline-controlled transactivator is driven by the regulatory elements of the CaMKIIa promoter (Jackson Labs—stock #007004) to restrict expression of the transactivator to forebrain neurons. Double transgenic (bi-genic) mice express mutant tau in forebrain neurons. The line has the ability to turn mutant tau expression off by administering doxycycline to mutant mice. To generate tau mutant mice with or without Hspa8^{G470R}, we will first introduce the chaperone variant into the responder line.

Mutant rTg4510 mice with or without Hspa8^{G470R} will then undergo a comprehensive battery of tests to determine if the chaperone variant mitigates tau pathology and disease severity in this model of dementia. A subgroup of rTg4510 mice administered doxycycline (200 ppm) in chow from 1.5 months of age will constitute our positive control. Doxycycline treatment suppresses mutant tau expression and thus prevents onset of brain pathology and disease. Briefly, the tests will be divided as follows.

Behavioral assays—rTg4510 model mice are reportedly hyperactive in open-field locomotor activity assays and exhibit significant deficits when challenged with spatial reference memory tasks as assessed in the Morris water maze. Accordingly, mutants with or without Hspa8^{G470R} will be tested in each of these assays. Animals will be assessed longitudinally at—a pre-symptomatic stage (1.5 months) and symptomatic stages (6 and 10 months). Mice that fail to complete the Morris water maze test over five trials will be excluded from the analysis.

Analysis of brain pathology—Argyrophilic tangle-like inclusions are a characteristic feature of the brains of bi-transgenic rTg4510 mice over the age of 4 months. Accordingly, we will begin by assessing tau burden in the cortex and hippocampus of rTg4510 mice with or without Hspa8^{G470R}. Since tau pathology is accompanied by neuronal loss and gross forebrain atrophy, we will also quantify neurons in the CA1 region of the hippocampus by stereology and examine brain size. Third, we will assess the extent of gliosis by using the astrocytic and microglial markers GFAP and Iba1 respectively; gliosis has been reported in rTg4510 mice as early as 2.5 months of age. Finally, we will determine if synaptic density which is reportedly reduced in rTg4510 mutants is normalized in rTg4510; Hspa8^{G470R} mice. We will accomplish this by quantifying dendritic spines on pyramidal neurons. In all instances, mice will be assessed at the time points indicated in the previous section following the completion of the behavioral assays.

Biochemical assays—We expect Hspa8^{G470R} to reduce levels of tau in brain tissue of the rTg4510 mice. We will test this by RTqPCR and by western blot analysis (for total tau and phosphorylated tau) on brain tissue from 1.5-month-old and 10-month-old rTg4510 mutants with and without Hspa8^{G470R}.

### Experiment 3—Does Viral-Mediated Expression of the Hspa8^{G470R} Variant Mitigate Disease in the Symptomatic rTg4510 model of AD?

The genetic experiments described above will determine if constitutive expression of Hspa8^{G470R} mitigates tau pathology and disease in the rTg4510 model of AD. Disease suppression can be achieved through targeting tau pathology by modulating Hsap8 chaperone activity. Therapy may also be initiated in the postnatal period. Consequently, in addition to the genetic experiments described above, we will deliver the Hspa8^{G470R} construct to bi-genic rTg4510 mutants in the AAV-PHP.eB or AAV9 vector, e.g., administered intravenously. Virus will be delivered to postnatal rTg4510 mutants at two time points-pre-symptomatically (1.5 months) and following symptom-onset (4.5 months). Mutants thus treated will then be analyzed as described in Expt. 2 except that the assessment will be carried out at 10 months. Efficiency of transduction will be gauged using a novel Hspa8^{G470R} specific antibody we have generated. The antibody robustly and selectively detects the G470R variant. As negative controls for the experiments in this part, we will use mutants injected with AAV-PHP.eB vector harboring a GFP cassette. As positive controls we will once again use rTg4510 mutants administered doxycycline beginning at 1.5 months of age.

### REFERENCES

1. Coovert, D.D., Le, T.T., McAndrew, P.E., Strasswimmer, J., Crawford, T.O., Mendell, J.R., Coulson, S.E., Androphy, E.J., Prior, T.W., and Burghes, A.H. (1997). The survival motor neuron protein in spinal muscular atrophy. Hum. Mol. Genet. 6, 1205-1214.
2. Lefebvre, S., Bürglen, L., Reboullet, S., Clermont, O., Burlet, P., Viollet, L., Benichou, B., Cruaud, C., Millasseau, P., and Zeviani, M. (1995). Identification and characterization of a spinal muscular atrophy-determining gene. Cell 80, 155-165.
3. Lefebvre, S., Burlet, P., Liu, Q., Bertrandy, S., Clermont, O., Munnich, A., Dreyfuss, G., and Melki, J. (1997). Correlation between severity and SMN protein level in spinal muscular atrophy. Nat. Genet. 16, 265-269.
4. Lorson, C.L., Hahnen, E., Androphy, E.J., and Wirth, B. (1999). A single nucleotide in the SMN gene regulates splicing and is responsible for spinal muscular atrophy. Proc. Natl. Acad. Sci. USA 96, 6307-6311.
5. Monani, U.R., Lorson, C.L., Parsons, D.W., Prior, T.W., Androphy, E.J., Burghes, A.H., and McPherson, J.D. (1999). A single nucleotide difference that alters splicing patterns distinguishes the SMA gene SMN1 from the copy gene SMN2. Hum. Mol. Genet. 8, 1177-1183.
6. Chaytow, H., Faller, K.M.E., Huang, Y.T., and Gillingwater, T.H. (2021). Spinal muscular atrophy: from approved therapies to future therapeutic targets for personalized medicine. Cell Rep. Med. 2, 100346.
7. Chaytow, H., Huang, Y.T., Gillingwater, T.H., and Faller, K.M.E. (2018). The role of survival motor neuron protein (SMN) in protein homeostasis. Cell. Mol. Life Sci. 75, 3877-3894.
8. Buchner, D.A., Trudeau, M., and Meisler, M.H. (2003). SCNM1, a putative RNA splicing factor that modifies disease severity in mice. Science 301, 967-969.
9. Nadeau, J.H. (2003). Modifier genes and protective alleles in humans and mice. Curr. Opin. Genet. Dev. 13, 290-295.
10. Dimitriadi, M., Sleigh, J.N., Walker, A., Chang, H.C., Sen, A., Kalloo, G., Harris, J., Barsby, T., Walsh, M.B., Satterlee, J.S., et al. (2010). Conserved genes act as modifiers of invertebrate SMN loss of function defects. PLOS Genet. 6, e1001172.
11. Sen, A., Dimlich, D.N., Guruharsha, K.G., Kankel, M.W., Hori, K., Yokokura, T., Brachat, S., Richardson, D., Loureiro, J., Sivasankaran, R., et al. (2013). Genetic circuitry of Survival motor neuron, the gene underlying spinal muscular atrophy. Proc. Natl. Acad. Sci. USA 110, E2371-E2380.
12. Hosseinibarkooie, S., Peters, M., Torres-Benito, L., Rastetter, R.H., Hupperich, K., Hoffmann, A., Mendoza-Ferreira, N., Kaczmarek, A., Janzen, E., Milbradt, J., et al. (2016). The power of human protective modifiers: PLS3 and CORO1C unravel impaired endocytosis in spinal muscular atrophy and rescue SMA phenotype. Am. J. Hum. Genet. 99, 647-665.
13. Janzen, E., Mendoza-Ferreira, N., Hosseinibarkooie, S., Schneider, S., Hupperich, K., Tschanz, T., Grysko, V., Riessland, M., Hammerschmidt, M., Rigo, F., et al. (2018). CHP1 reduction ameliorates spinal muscular atrophy pathology by restoring calcineurin activity and endocytosis. Brain 141, 2343-2361.
14. Riessland, M., Kaczmarek, A., Schneider, S., Swoboda, K.J., Löhr, H., Bradler, C., Grysko, V., Dimitriadi, M., Hosseinibarkooie, S., Torres-Benito, L., et al. (2017). Neurocalcin Delta suppression protects against spinal muscular atrophy in humans and across species by restoring impaired endocytosis. Am. J. Hum. Genet. 100, 297-315.
15. Oprea, G.E., Kröber, S., McWhorter, M.L., Rossoll, W., Müller, S., Krawczak, M., Bassell, G.J., Beattie, C.E., and Wirth, B. (2008). Plastin 3 is a protective modifier of autosomal recessive spinal muscular atrophy. Science 320, 524-527.
16. Le, T.T., Pham, L.T., Butchbach, M.E., Zhang, H.L., Monani, U.R., Coovert, D.D., Gavrilina, T.O., Xing, L., Bassell, G.J., and Burghes, A.H. (2005). SMNDelta7, the major product of the centromeric survival motor neuron (SMN2) gene, extends survival in mice with spinal muscular atrophy and associates with full-length SMN. Hum. Mol. Genet. 14, 845-857.
17. Crawford, T.O., and Pardo, C.A. (1996). The neurobiology of childhood spinal muscular atrophy. Neurobiol. Dis. 3, 97-110.
18. Harding, B.N., Kariya, S., Monani, U.R., Chung, W.K., Benton, M., Yum, S.W., Tennekoon, G., and Finkel, R.S. (2015). Spectrum of neuropathophysiology in spinal muscular atrophy type I. J. Neuropathol. Exp. Neurol. 74, 15-24.
19. Ramos, D.M., d'Ydewalle, C., Gabbeta, V., Dakka, A., Klein, S.K., Norris, D.A., Matson, J., Taylor, S.J., Zaworski, P.G., Prior, T.W., et al. (2019). Age-dependent SMN expression in disease-relevant tissue and implications for SMA treatment. J. Clin. Invest. 129, 4817-4831.
20. Kariya, S., Park, G.H., Maeno-Hikichi, Y., Leykekhman, O., Lutz, C., Arkovitz, M.S., Landmesser, L.T., and Monani, U.R. (2008). Reduced SMN protein impairs maturation of the neuromuscular junctions in mouse models of spinal muscular atrophy. Hum. Mol. Genet. 17, 2552-2569.
21. Kong, L., Wang, X., Choc, D.W., Polley, M., Burnett, B.G., Bosch-Marcé, M., Griffin, J.W., Rich, M.M., and Sumner, C.J. (2009). Impaired synaptic vesicle release and immaturity of neuromuscular junctions in spinal muscular atrophy mice. J. Neurosci. 29, 842-851.
22. Murray, L.M., Comley, L.H., Thomson, D., Parkinson, N., Talbot, K., and Gillingwater, T.H. (2008). Selective vulnerability of motor neurons and dissociation of pre- and post-synaptic pathology at the neuromuscular junction in mouse models of spinal muscular atrophy. Hum. Mol. Genet. 17, 949-962.
23. Heier, C.R., Satta, R., Lutz, C., and DiDonato, C.J. (2010). Arrhythmia and cardiac defects are a feature of spinal muscular atrophy model mice. Hum. Mol. Genet. 19, 3906-3918.
24. Woo, J., Kwon, S.K., Nam, J., Choi, S., Takahashi, H., Krueger, D., Park, J., Lee, Y., Bac, J.Y., Lee, D., et al. (2013). The adhesion protein IgSF9b is coupled to neuroligin 2 via S-SCAM to promote inhibitory synapse development. J. Cell Biol. 201, 929-944.
25. Tobaben, S., Thakur, P., Fernandez-Chacón, R., Südhof, T.C., Rettig, J., and Stahl, B. (2001). A trimeric protein complex functions as a synaptic chaperone machine. Neuron 31, 987-999.
26. Zinsmaier, K.E., and Bronk, P. (2001). Molecular chaperones and the regulation of neurotransmitter exocytosis. Biochem. Pharmacol. 62, 1-11.
27. Jodelka, F.M., Ebert, A.D., Duelli, D.M., and Hastings, M.L. (2010). A feedback loop regulates splicing of the spinal muscular atrophy-modifying gene, SMN2. Hum. Mol. Genet. 19, 4906-4917.
28. Ruggiu, M., McGovern, V.L., Lotti, F., Saieva, L., Li, D.K., Kariya, S., Monani, U.R., Burghes, A.H., and Pellizzoni, L. (2012). A role for SMN exon 7 splicing in the selective vulnerability of motor neurons in spinal muscular atrophy. Mol. Cell. Biol. 32, 126-138.
29. Monani, U.R., Sendtner, M., Coovert, D.D., Parsons, D.W., Andreassi, C., Le, T.T., Jablonka, S., Schrank, B., Rossoll, W., Prior, T.W., et al. (2000). The human centromeric survival motor neuron gene (SMN2) rescues embryonic lethality in Smn(−/−) mice and results in a mouse with spinal muscular atrophy. Hum. Mol. Genet. 9, 333-339.
30. Lotti, F., Imlach, W.L., Saieva, L., Beck, E.S., Hao, le T., Li, D.K., Jiao, W., Mentis, G.Z., Beattie, C.E., McCabe, B.D., et al. (2012). An SMN-dependent U12 splicing event essential for motor circuit function. Cell 151, 440-454.
31. Tisdale, S., Lotti, F., Saieva, L., Van Meerbeke, J.P., Crawford, T.O., Sumner, C.J., Mentis, G.Z., and Pellizzoni, L. (2013). SMN is essential for the biogenesis of U7 small nuclear ribonucleoprotein and 30-end formation of histone mRNAs. Cell Rep. 5, 1187-1195.
32. Südhof, T.C., and Rizo, J. (2011). Synaptic vesicle exocytosis. Cold Spring Harb. Perspect. Biol. 3.
33. Uytterhoeven, V., Lauwers, E., Maes, I., Miskiewicz, K., Melo, M.N., Swerts, J., Kuenen, S., Wittocx, R., Corthout, N., Marrink, S.J., et al. (2015). Hsc70-4 deforms membranes to promote synaptic protein turnover by endosomal microautophagy. Neuron 88, 735-748.
34. Chandra, S., Gallardo, G., Ferna'ndez-Chaco'n, R., Schl€uter, O.M., and S€udhof, T.C. (2005). Alpha-synuclein cooperates with CSPalpha in preventing neurodegeneration. Cell 123, 383-396.
35. Sharma, M., Burré, J., and Südhof, T.C. (2011). CSPalpha promotes SNARE-complex assembly by chaperoning SNAP-25 during synaptic activity. Nat. Cell Biol. 13, 30-39.
36. Bowerman, M., Anderson, C.L., Beauvais, A., Boyl, P.P., Witke, W., and Kothary, R. (2009). SMN, profilin Ila and plastin 3: a link between the deregulation of actin dynamics and SMA pathogenesis. Mol. Cell. Neurosci. 42, 66-74.
37. Ripps, M.E., Huntley, G.W., Hof, P.R., Morrison, J.H., and Gordon, J.W. (1995). Transgenic mice expressing an altered murine superoxide dismutase gene provide an animal model of amyotrophic lateral sclerosis. Proc. Natl. Acad. Sci. USA 92, 689-693.
38. Kim, J.K., and Monani, U.R. (2018). Augmenting the SMN protein to treat infantile spinal muscular atrophy. Neuron 97, 1001-1003.
39. Wilhelm, B.G., Mandad, S., Truckenbrodt, S., Kröhnert, K., Schäfer, C., Rammner, B., Koo, S.J., Claβen, G.A., Krauss, M., Haucke, V., et al. (2014). Composition of isolated synaptic boutons reveals the amounts of vesicle trafficking proteins. Science 344, 1023-1028.
40. Liu, T., Daniels, C.K., and Cao, S. (2012). Comprehensive review on the HSC70 functions, interactions with related molecules and involvement in clinical diseases and therapeutic potential. Pharmacol. Ther. 136, 354-374.
41. Barbosa-Morais, N.L., Carmo-Fonseca, M., and Aparício, S. (2006). Systematic genome-wide annotation of spliceosomal proteins reveals differential gene family expansion. Genome Res. 16, 66-77.
42. Rappsilber, J., Ryder, U., Lamond, A.I., and Mann, M. (2002). Large-scale proteomic analysis of the human spliceosome. Genome Res. 12, 1231-1245.
43. Zheng, S., Damoiseaux, R., Chen, L., and Black, D.L. (2013). A broadly applicable high-throughput screening strategy identifies new regulators of Dlg4 (Psd-95) alternative splicing. Genome Res. 23, 998-1007.
44. Zhu, X., Zhao, X., Burkholder, W.F., Gragerov, A., Ogata, C.M., Gottesman, M.E., and Hendrickson, W.A. (1996). Structural analysis of substrate binding by the molecular chaperone DnaK. Science 272, 1606-1614.
45. Khan, S., and Vihinen, M. (2007). Spectrum of disease-causing mutations in protein secondary structures. BMC Struct. Biol. 7, 56.
46. Molnár, J., Szaka'cs, G., and Tusnády, G.E. (2016). Characterization of disease-associated mutations in human transmembrane proteins. PLOS One 11, e0151760.
47. Shen, L., and Ji, H.F. (2015). Mutational spectrum analysis of neurodegenerative diseases and its pathogenic implication. Int. J. Mol. Sci. 16, 24295-24301.
48. Taylor, I.R., Ahmad, A., Wu, T., Nordhues, B.A., Bhullar, A., Gestwicki, J.E., and Zuiderweg, E.R.P. (2018). The disorderly conduct of Hsc70 and its interaction with the Alzheimer's-related Tau protein. J. Biol. Chem. 293, 10796-10809.
49. Meister, G., Bühler, D., Pillai, R., Lottspeich, F., and Fischer, U. (2001). A multiprotein complex mediates the ATP-dependent assembly of spliceosomal U snRNPs. Nat. Cell Biol. 3, 945-949.
50. Ryu, S.W., Stewart, R., Pectol, D.C., Ender, N.A., Wimalarathne, O., Lee, J.H., Zanini, C.P., Harvey, A., Huibregtse, J.M., Mueller, P., et al. (2020). Proteome-wide identification of HSP70/HSC70 chaperone clients in human cells. PLOS Biol. 18, e3000606.
51. Shafey, D., Boyer, J.G., Bhanot, K., and Kothary, R. (2010). Identification of novel interacting protein partners of SMN using tandem affinity purification. J. Proteome Res. 9, 1659-1669.
52. Garcera, A., Bahi, N., Periyakaruppiah, A., Arumugam, S., and Soler, R.M. (2013). Survival motor neuron protein reduction deregulates autophagy in spinal cord motoneurons in vitro. Cell Death Dis. 4, e686.
53. Takikawa, K., and Nishimune, H. (2022). Similarity and diversity of presynaptic molecules at neuromuscular junctions and central synapses. Biomolecules 12, 179.
54. Varoqueaux, F., Sigler, A., Rhee, J.S., Brose, N., Enk, C., Reim, K., and Rosenmund, C. (2002). Total arrest of spontaneous and evoked synaptic transmission but normal synaptogenesis in the absence of Munc13-mediated vesicle priming. Proc. Natl. Acad. Sci. USA 99, 9037-9042.
55. Varoqueaux, F., Sons, M.S., Plomp, J.J., and Brose, N. (2005). Aberrant morphology and residual transmitter release at the Munc 13-deficient mouse neuromuscular synapse. Mol. Cell. Biol. 25, 5973-5984.
56. Dong, W., Radulovic, T., Goral, R.O., Thomas, C., Suarez Montesinos, M., Guerrero-Given, D., Hagiwara, A., Putzke, T., Hida, Y., Abe, M., et al. (2018). CAST/ELKS proteins control voltage-gated Ca2+ channel density and synaptic release probability at a mammalian central synapse. Cell Rep. 24, 284-293.e6.
57. Tokoro, T., Higa, S., Deguchi-Tawarada, M., Inoue, E., Kitajima, I., and Ohtsuka, T. (2007). Localization of the active zone proteins CAST, ELKS, and Piccolo at neuromuscular junctions. NeuroReport 18, 313-316.
58. Coyne, A.N., Lorenzini, I., Chou, C.C., Torvund, M., Rogers, R.S., Starr, A., Zaepfel, B.L., Levy, J., Johannesmeyer, J., Schwartz, J.C., et al. (2017). Post-transcriptional inhibition of Hsc70-4/HSPA8 expression leads to synaptic vesicle cycling defects in multiple models of ALS. Cell Rep. 21, 110-125.
59. Michaud, M., Arnoux, T., Bielli, S., Durand, E., Rotrou, Y., Jablonka, S., Robert, F., Giraudon-Paoli, M., Riessland, M., Mattei, M.G., et al. (2010). Neuromuscular defects and breathing disorders in a new mouse model of spinal muscular atrophy. Neurobiol. Dis. 38, 125-135.
60. Feng, Z., Ling, K.K., Zhao, X., Zhou, C., Karp, G., Welch, E.M., Naryshkin, N., Ratni, H., Chen, K.S., Metzger, F., et al. (2016). Pharmacologically induced mouse model of adult spinal muscular atrophy to evaluate effectiveness of therapeutics after disease onset. Hum. Mol. Genet. 25, 964-975.
61. Iyer, C.C., Corlett, K.M., Massoni-Laporte, A., Duque, S.I., Madabusi, N., Tisdale, S., McGovern, V.L., Le, T.T., Zaworski, P.G., Arnold, W.D., et al. (2018). Mild SMN missense alleles are only functional in the presence of SMN2 in mammals. Hum. Mol. Genet. 27, 3404-3416.
62. Li, H., and Durbin, R. (2009). Fast and accurate short read alignment with Burrows-Wheeler transform. Bioinformatics 25, 1754-1760.
63. Li, H., Handsaker, B., Wysoker, A., Fennell, T., Ruan, J., Homer, N., Marth, G., Abecasis, G., and Durbin, R.; 1000 Genome Project Data Processing Subgroup (2009). The Sequence Alignment/Map format and SAMtools. Bioinformatics 25, 2078-2079.
64. Wang, K., Li, M., and Hakonarson, H. (2010). ANNOVAR: functional annotation of genetic variants from high-throughput sequencing data. Nucleic Acids Res. 38, e164.
65. Park, G.H., Maeno-Hikichi, Y., Awano, T., Landmesser, L.T., and Monani, U.R. (2010). Reduced survival of motor neuron (SMN) protein in motor neuronal progenitors functions cell autonomously to cause spinal muscular atrophy in model mice expressing the human centromeric (SMN2) gene. J. Neurosci. 30, 12005-12019.
66. Ling, K.K., Lin, M.Y., Zingg, B., Feng, Z., and Ko, C.P. (2010). Synaptic defects in the spinal and neuromuscular circuitry in a mouse model of spinal muscular atrophy. PLOS One 5, e15457.
67. Ruiz, R., Casañas, J.J., Torres-Benito, L., Cano, R., and Tabares, L. (2010). Altered intracellular Ca2+ homeostasis in nerve terminals of severe spinal muscular atrophy mice. J. Neurosci. 30, 849-857.
68. Caine, C., Shohat, M., Kim, J.K., Nakanishi, K., Homma, S., Mosharov, E.V., and Monani, U.R. (2017). A pathogenic S250F missense mutation results in a mouse model of mild aromatic L-amino acid decarboxylase (AADC) deficiency. Hum. Mol. Genet. 26, 4406-4415.
69. Kariya, S., Obis, T., Garone, C., Akay, T., Sera, F., Iwata, S., Homma, S., and Monani, U.R. (2014). Requirement of enhanced Survival Motoneuron protein imposed during neuromuscular junction maturation. J. Clin. Invest. 124, 785-800.
70. Maury, Y., Côme, J., Piskorowski, R.A., Salah-Mohellibi, N., Chevaleyre, V., Peschanski, M., Martinat, C., and Nedelec, S. (2015). Combinatorial analysis of developmental cues efficiently converts human pluripotent stem cells into multiple neuronal subtypes. Nat. Biotechnol. 33, 89-96.
71. Sison, S.L., Patitucci, T.N., Seminary, E.R., Villalon, E., Lorson, C.L., and Ebert, A.D. (2017). Astrocyte-produced miR-146a as a mediator of motor neuron loss in spinal muscular atrophy. Hum. Mol. Genet. 26, 3409-3420.

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description and accompanying drawings is offered by way of illustration only and not as a limitation.

What is claimed is:

1. A method of treating a neurodegenerative disease in a subject, the method comprising administering an effective amount of a nucleic acid molecule encoding a variant, mutant or modulator of heat shock 70kDa protein 8 (Hspa8) to the subject.

2. A method of treating a neurodegenerative disease in a subject, the method comprising administering an effective amount of a variant, mutant or modulator of heat shock 70 kDa protein 8 (Hspa8) of Hsa8 to the subject.

3. The method of claim 1 or 2, wherein the mutant of Hspa8 comprises a missense mutation.

4. The method of claim 1 or 2, wherein the variant of Hspa8 is Hspa8^{G470R}.

5. The method of claim 1 or 2, wherein the mutant of Hspa8 comprises a mutation in a substrate binding domain of Hspa8.

6. The method of claim 1 or 2, wherein the mutant of Hspa8 comprises a mutation in an ATPase domain of Hspa8.

7. The method of claim 1 or 2, wherein the variant or mutant of Hspa8 has a lower chaperone activity than wildtype Hspa8.

8. The method of claim 1 or 2, wherein the variant or mutant of Hspa8 has a greater microautophagy activity than wildtype Hspa8.

9. The method of any one of claims 1 and 3-8, wherein the nucleic acid molecule comprises a recombinant adeno-associated virus (AAV) vector.

10. The method of claim 9, wherein the AAV vector is AAV-PHP.eB or AAV9.

11. The method of any one of claims 1 and 3-10, wherein the nucleic acid molecule is administered to the central nervous system (CNS) of the subject.

12. The method of any one of claims 1 and 3-11, wherein the nucleic acid molecule is administered to the spinal cord of the subject.

13. The method of any one of claims 1 and 3-12, wherein the nucleic acid molecule is administered by intrathecal injection.

14. The method of any one of claims 1 and 3-10, wherein the nucleic acid molecule is administered orally, intravenously, intramuscularly, topically, arterially, or subcutaneously.

15. The method of any of the previous claims, wherein the modulator binds to a substrate binding domain of Hspa8.

16. The method of any of the previous claims, wherein the modulator binds to an ATPase domain of Hspa8.

17. The method of any of the previous claims, wherein the modulator decreases a chaperone activity of Hspa8.

18. The method of any of the previous claims, wherein the modulator increases a microautophagy activity of Hspa8.

19. The method of any of the previous claims, wherein the modulator is an inhibitor of Hsa8.

20. The method of any of the previous claims, wherein the modulator is a small molecule, a polynucleotide, or an antibody or antigen-binding portion thereof.

21. The method of claim 20, wherein the polynucleotide is a small interfering RNA (siRNA) or an antisense molecule.

22. The method of any of the previous claims, wherein the modulator comprises a CRISPR/Cas system.

23. The method of any one of claims 2-22, wherein the variant, mutant or modulator is administered to the central nervous system (CNS) of the subject.

24. The method of any one of claims 2-23, wherein the variant, mutant or modulator is administered to the spinal cord of the subject.

25. The method of any one of claims 2-24, wherein the variant, mutant or modulator is administered by intrathecal injection.

26. The method of any one of claims 2-22, wherein the variant, mutant or modulator is administered orally, intravenously, intramuscularly, topically, arterially, or subcutaneously.

27. The method of any of the previous claims, wherein the neurodegenerative disease is a tau-related disease or tauopathy.

28. The method of claim 27, wherein the tau-related disease or tauopathy is Alzheimer's disease (AD), primary age-related tauopathy (PART) dementia, chronic traumatic encephalopathy (CTE), progressive supranuclear palsy (PSP), corticobasal degeneration (CBD), frontotemporal dementia and parkinsonism linked to chromosome 17 (FTDP-17), vacuolar tauopathy, lytico-bodig disease (Parkinson-dementia complex of Guam), Ganglioglioma and gangliocytoma, meningioangiomatosis, postencephalitic parkinsonism, subacute sclerosing panencephalitis (SSPE), lead encephalopathy, tuberous sclerosis, pantothenate kinase-associated neurodegeneration, lipofuscinosis, Pick's disease, corticobasal degeneration, argyrophilic grain disease (AGD), spinal muscular atrophy (SMA) or amyotrophic lateral sclerosis (ALS).

29. The method of any of the previous claims, wherein the neurodegenerative disease is amyotrophic lateral sclerosis, multiple sclerosis, Parkinson's disease, Alzheimer's disease, Huntington's disease, multiple system atrophy, Batten disease, or a prion disease.

30. The method of any of the previous claims, wherein the neurodegenerative disease is a neurodegenerative dementia.

31. The method of any of the previous claims, wherein the neurodegenerative disease is a tau-related dementia.

32. The method of any of the previous claims, further comprising administering a SMN2 splicing modifier to the subject.

33. The method of any of the previous claims, wherein the subject is a mammal.

34. The method of claim 31, wherein the mammal is a human, a rodent, or a simian.

35. The method of claim 31, wherein the mammal is a human.


SEQUENCE LISTING (3 sequences)

SEQ ID NO: 1 - protein, 10 aa, Homo sapiens
IPPAPRGVPQ

SEQ ID NO: 2 - DNA, 159 bp, synthetic construct
  source 1..159: mol_type = other DNA
tatgaaggtgaaagggccatgaccaaggacaacaacctgcttggaaagttcgagctcacaggcatccctccagcaccccgtcgggtccctcagattgaggttacttttgacatcgatgccaatggcatcctcaatgtttctgctgtagataagagcaca

SEQ ID NO: 3 - RNA, 35 nt, synthetic construct
  source 1..35: mol_type = other RNA
gcatccctccagcaccccggttttagagctatgct